(19) United States
(12) Patent Application Publication
(10) Pub. No.: US 2005/0028229 A1
(43) Pub. Date: Feb. 3, 2005

(54) Alpha-hemoglobin stabilizing protein transgenic mouse and methods of use thereof

(76) Inventors: Mitchell Weiss, Wynnewood, PA (US); Gerd Blobel, Merion, PA (US); Yi Kong, Philadelphia, PA (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/824,448

(22) Filed: Apr. 14, 2004

Related U.S. Application Data
(60) Provisional application No. 60/462,771, filed on Apr. 14, 2003.
(60) Provisional application No. 60/477,991, filed on Jun. 12, 2003.

(51) Int. Cl. A01K 67/027; C12N 5/06

(52) U.S. Cl. 800/18; 435/354

(57) ABSTRACT

A transgenic non-human animal with alterations in the Alpha Hemoglobin Stabilizing Protein (AHSP) gene is prepared by introduction of a gene encoding an altered Alpha Hemoglobin Stabilizing Protein (AHSP) protein into a host non-human animal. Methods for using transgenic mice so generated to screen for agents that effect Alpha Hemoglobin Stabilizing Protein (AHSP)'s hemoglobin binding activity are also provided.

### CONTINUING APPLICATION DATA

This application claims priority under 35 U.S.C. §119 (e) to U.S. Provisional Patent Application Nos. 60/462,771 filed on Apr. 14, 2003, and 60/477,991, filed on Jun. 12, 2003, the entire disclosures of which are incorporated by reference herein.

Pursuant to 35 U.S.C. §202(c), it is acknowledged that the U.S. Government has certain rights in the invention described herein, which was made in part with funds from the National Institute of Health, grant No. NIH-NIDDK R01DK61692.

### FIELD OF THE INVENTION

This invention relates the fields of recombinant DNA technology, transgenic animals, and spongiform pathologies. More specifically, a transgenic nonhuman animal is provided comprising an altered Alpha Hemoglobin Stabilizing Protein (AHSP) gene. Methods of using such animals to identify therapeutic agents useful for the treatment of anemias and spongiform encephalopathies are also provided.

### BACKGROUND OF THE INVENTION

Several publications are cited in this application by author name, year and journal of publication in parentheses in order to more fully describe the state of the art to which this invention pertains. Several patents are also cited throughout the specification. The disclosure of each of these citations is incorporated by reference herein.

Erythroid Differentiation Related Factor (EDRF) is a 102 amino acid protein which is highly conserved in humans, pigs, cows, and rats. EDRF is expressed exclusively in hematopoietic tissues, and has been linked to numerous blood and neurological disorders in mice (Kihm et al., (2002) Nature 417, pages 758-763.)

Recent research indicates that EDRF is differentially expressed in the blood and bone marrow of animals infected with TSE's. TSE's or Transmissible Spongiform Encephalopathies are a group of fatal neurodegenerative disorders which include scrapie in sheep, Bovine Spongiform Encephalopathy (BSE) in cattle, Chronic Wasting Disease in deer and kuru, Creutzfeldt-Jakob Disease (CJD) and variant CJD in humans. These disorders all exhibit a short clinical course, after a long incubation period characterized by a lack of any immune response. Thus, early detection of Transmissible Spongiform Encephalopathies is a particular challenge. Currently, the only test for TSE's is post-mortem analysis of brain tissue (Roslin Institute, Edinburgh, (2001) Annual Report 00-01, pp22-25).

The development and progression of TSE's is correlated with the presence of protein-like molecules called prions (PrP). The cellular form of PrP is soluble, sensitive to proteinase K digestion, mostly composed of alpha helices, and is non-infectious. An infectious, protease resistant isoform may occur by spontaneous mutation, or by infection. This isoform tends to convert normal PrP to infectious, protease resistant PrP. Eventually, these prions can accumulate to a level where plaque-type structures form, leading to neuronal dysfunction and damage (Roslin Institute, Edinburgh, (2001) Annual Report 00-01, pp 22-25).

A potential new diagnostic marker for TSE's was discovered by a group at the Roslin Institute in Scotland. Gene expression and protein profiles from Scrapie infected mice were compared with healthy mice. A single gene was found to be differentially expressed, representing a transcript which had previously been designated Erythroid Differentiation Related Factor (EDRF). Further EDRF expression analysis was conducted in mice and in humans. In mice EDRF is expressed in blood, bone marrow, and spleen, while in humans EDRF expression is limited to the bone marrow and blood. These results also confirmed that EDRF levels are lower in the blood and bone marrow of TSE infected animals (Roslin Institute, Edinburgh, (2001) Annual Report 00-01, pp22-25).

While the biological function of EDRF has not been totally elucidated, it has been reported that the molecule is expressed exclusively in hematopoietic cells of erythroid lineage, suggesting a role in blood cell development. Additionally, there appears to be a definitive link between EDRF down regulation and Transmissible Spongiform Encephalopathies.

The development of red blood cells (erythrocytes) is distinguished by the production of the oxygen carrier, hemoglobin A (HbA), a heterotetramer of α- and β-hemoglobin subunits. (Kihm et al., (2002) Nature 417:758-763). Two α- and two β-globulin chains make up a HbA molecule, each containing a heme, which is a non-protein group that carries oxygen. The alpha (α-) and beta (β-) globulin chains are encoded by distinct genes, which are assembled to form the HbA molecule. Proper assembly of the HbA molecule is essential, not only to create a functional hemoglobin supply, but also to prevent deleterious physiological effects which result from misassembly. For example, the U-chain can associate on it's own with heme and form a tetramer called HbH. Although this tetramer will bind oxygen, it does not release it, and thus is not a functional hemoglobin tetramer. Also, heme bound α-chains form precipitates, called α-inclusion bodies that damage red blood cells (Luzzatto et al., (2002) Nature 417:703-705).

There are many disorders that result in a misassembly of the HbA molecule. β-thalassemia is characterized by a mutant β-globulin gene. The severity of the disease depends on the ratio of α-hemoglobin levels to β-hemoglobin levels. Homozygous β-thalassemia results in a complete lack of production of the β-globulin chain, and a resultant excess of α-globulin chains. Intact monomeric α-globulin (α-Hb) generates reactive oxygen species (ROS) that damage cellular proteins, lipids and nucleic acids (Brunori, M., et al. (1974) Eur. J. Biochem., 53, 99-104). In addition, α Hb is structurally unstable with a tendency to denature upon oxidation, filling the cytoplasm and cell membrane with precipitated a globin polypeptides, free heme, porphyrins and iron which further propagate ROS production (reviewed in Shinar, E. and Rachmilewitz, E. A. (1990) Semin Hematol, 27, 70-82). Together, these effects reduce the lifespan of circulating erythrocytes and also impair the viability of erythroid precursors in hematopoietic tissues, causing ineffective erythropoiesis. Patients with homozygous β-thalassemia exhibit severe anemia, requiring transfusions or a bone marrow transplant. Heterozygous carriers of β-thalassemia, are essentially asymptomatic, unless they have a triplicated α-globulin gene. Patients with β-thalassemia major who also have a mutated α-globulin gene usually have a milder condition.

Most cells contain compensatory mechanisms to cope with unstable proteins (Wickner, S., et al. (1999) Science, 286, 1888-1893). These include molecular chaperones that stabilize proteins and in some cases facilitate their folding into native structures. In addition, there are degradation pathways that recognize and eliminate improperly folded polypeptides. Accordingly, tissues typically tolerate some protein instability, with disease ensuing only when the compensatory mechanisms become overwhelmed. Several findings indicate that mechanisms to neutralize free α Hb exist in erythroid cells. First, erythroid precursors contain a small pool of excess free α Hb with no apparent ill effects (Shaeffer, J. R. (1967) Biochemical & Biophysical Research Communications, 28, 647-652; Tavill, A. S., et al. (1967) Transactions of the Association of American Physicians, 80, 305-313). In addition, erythropoiesis is relatively normal in individuals lacking one functional β globin gene (β thalassemia trait). Finally, there is frequent unexplained phenotypic diversity among individuals with the same β thalassemia genotype (reviewed in Weatherall, D. J. (2001) Nat Rev Genet, 2, 245-255). The latter observation could be explained by genetic variations in processes that stabilize or eliminate free α Hb. These mechanisms are now beginning to be elucidated in erythroid cells.

GATA-1 is a DNA binding protein that is essential for survival and maturation of lineage committed erythroid precursors, and controls many genes and metabolic pathways that participate in hemoglobin synthesis. As is disclosed herein, EDRF mRNA and protein were found to be rapidly induced by GATA-1. In light of this discovery, it is proposed that EDRF be renamed Alpha Hemoglobin Stabilizing Protein.

Accordingly, a need exists in the art to further characterize EDRF/AHSP's function and it's link to transmissible spongiform encephalopathies, β-thalassemias and anemias.

### SUMMARY OF THE INVENTION

This invention provides non-human transgenic animals in which the Alpha Hemoglobin Stabilizing Protein (AHSP) gene has been altered, and methods of use thereof. The AHSP transgenic mice of the invention are fertile and display grossly normal growth and development. AHSP^{-/-} mice exhibit defective hemoglobin metabolism in vivo, characterized by a reduced erythrocyte half-life, abnormal erythrocyte morphology (similar to that seen in β-thalassemic mice), and denatured hemoglobin inclusions (Heinz bodies). Accordingly, AHSP^{-/-} mice provide an ideal in vivo system for study of hemoglobin assembly. Further, AHSP overexpressing mice are also exemplary in vivo models for the study of hemoglobin assembly.

In a preferred embodiment of the invention, mice transgenic for the AHSP gene are provided. Such mice (AHSP^{-/-} and AHSP overexpressers) may be used to advantage in assays for the identification of therapeutic agents useful for the diagnosis and treatment of disorders such as β-thalassemia, anemia, transmissible spongiform encephalopathies (TSE's) such as bovine spongiform encephalopathy (BSE), Alzheimer's disease, and other disorders which are characterized by misassembly of hemoglobin, or the formation of plaques or inclusion bodies.

In yet another embodiment of the invention assays are provided for diagnosis of pathologies such as β-thalassemia, anemia, TSE's, Alzheimer's disease, and other disorders which are characterized by misassembly of hemoglobin, or the formation of plaques or inclusion bodies. An exemplary assay entails analysis of expression levels of the AHSP gene and characterization of its protein product. Another exemplary assay includes detecting a prion disease such as BSE in an animal (e.g. bovine, ovine, porcine, and so forth) by detecting a decreased level of AHSP.

The invention also provides methods for the treatment of pathologies such as β-thalassemia, anemia, TSE's Alzheimer's disease, and other disorders which are characterized by misassembly of hemoglobin, or the formation of plaques or inclusion bodies. An exemplary method of gene therapy is provided which replaces or supplements a defective AHSP gene.

In yet another aspect, the invention provides methods for screening therapeutic agents for the treatment or amelioration of β-thalassemia, anemia, TSE's, Alzheimer's disease, and other disorders which are characterized by misassembly of hemoglobin, or the formation of plaques or inclusion bodies. An exemplary method entails exposing AHSP defective or deficient cells or animals to a putative therapeutic agent and determining the effect of the agent on test cells (ie: is AHSP activity increased or improved).

In another aspect of the invention, methods of generating antibodies using the alpha hemoglobin stabilizing protein knockout mice of the invention are provided. Additionally, the antibodies produced by this method are provided.

Also encompassed by the present invention are kits for the diagnosis and/or treatment of disorders such as β-thalassemia, anemia, TSE's such as BSE, and Alzheimer's disease.

### BRIEF DESCRIPTION OF THE DRAWINGS

FIGS. 1a-1d show that AHSP is a GATA-1-induced erythroid-specific gene. FIG. 1a shows a Northern blot probed with full length AHSP complementary DNA. G1E and G1E-ER2 cells were treated with β-oestradiol (E2) or the partial oestrogen receptor agonist tamoxifen. G1E is a GATA-1⁻ erythroid line that undergoes GATA-1-dependent terminal maturation. G1E-ER2 cells are a G1E subclone engineered to express an oestradiol-activated form of GATA-1. Total RNA per lane, 15 μg. FIG. 1b shows a Western blot of β-oestradiol-treated G1E-ER2 cells with anti-murine AHSP antibody. Total cellular protein per lane, 10 μg. FIG. 1c is a multi-tissue northern blot with full-length AHSP probe (15 μg total RNA per lane). e12.5 is day 12.5 mouse embryo; Li is adult liver; K is kidney; Te is testis; H is heart; BM is bone marrow; Th is thymus; Sp is spleen; FL is fetal liver; Br is brain. FIG. 1d shows RT-PCR analysis of AHSP expression in primary hematopoietic colonies. EryD is short for definitive (adult-type) erythroid colonies derived from wildtype (WT) or GATA-1⁻ (G-1⁻) embryonic stem cells; ssDNA is salmon sperm DNA; GM is granulocyte-macrophage; Mega is megakaryocyte; Mac is macrophage. L-32 is a constitutively expressed ribosomal protein. For comparison, samples were analyzed from β-oestradiol (E2)-treated G1E-ER2 cells.

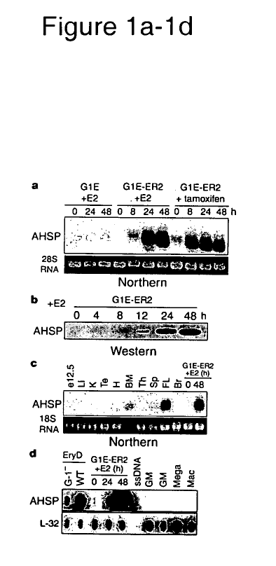

FIGS. 2a-2d show that AHSP binds free α-hemoglobin but not β-hemoglobin. FIG. 2a is a gel showing the results from a GST-AHSP pulldown analysis. AHSP-GST or GST crosslinked to agarose beads were incubated with extracts from MEL cells. Bound protein was resolved on a 12.5% SDS-polyacrylamide gel and detected with Coomassie blue. U is uninduced; I is DMSO-induced for 72 h. The prominent bound protein (indicated with an arrow) was determined as being α-hemoglobin by Edman sequence analysis. FIG. 2b is a chromatogram of mixtures containing equimolar ratios of recombinant human AHSP and purified human carboxy (CO)-α- or β hemoglobins analyzed by gel filtration. AHSP (top panel) was detected by light absorbance at 280 nm; hemoglobins were detected by absorbance at 540 nm. Arrows indicate molecular size standards (shown as 10⁻³× molecular masses). In solution, purified α-hemoglobin exists mainly as a monomer (Mᵣ ˜16K);β-hemoglobin exists mainly as a tetramer (HbH, Mᵣ˜64K) . The retention time observed for AHSP predicts a Mᵣ of approximately 35K, significantly greater than the 12K predicted by the primary amino acid sequence. This anomaly could be a consequence of either multimerization or an asymmetric shape. FIG. 2c shows results obtained from cellulose acetate electrophoresis. AHSP was incubated with purified CO-α- or CO-β-hemoglobin and analyzed. Duplicate strips were stained with benzidine to identify heme-containing proteins, and/or with Ponceau S to stain all proteins. Concentrations of AHSP and hemoglobins (μM) are indicated at the top. Bands a-d were eluted from the Ponceau-S-stained cellulose acetate strip and analyzed by western blotting with anti-AHSP antibody (bottom panel). Extract from oestradiol-treated G1E-ER2 cells (Ctl) served as a positive control for AHSP protein. FIG. 2d is a blot showing that AHSP and β-hemoglobin compete for α-hemoglobin binding. AHSP and CO-α-hemoglobin were preincubated for 1 h, followed by addition of CO-β-hemoglobin at the indicated concentrations (μM) and incubation for an additional 30 min. Cellulose acetate electrophoresis and western blotting were performed as described in panel 2c.

FIGS. 3a-3d show that AHSP prevents oxidant-induced precipitation of α-hemoglobin in solution. In FIG. 3a, human AHSP was preincubated with purified oxygenated (oxy)α-hemoglobin at the indicated molar ratios for 60 min. Potassium ferricyanide (50 μM final concentration) was added at zero time to induce heme oxidation. Protein precipitation was monitored by light scattering at 700 nm. In FIG. 3b, α-Hemoglobin reactions from FIG. 3a were centrifuged 60 min after the addition of ferricyanide. Soluble α-hemoglobin remaining in the supernatant was quantified by light absorbance at 540 nm. The y-axis shows the fraction of the original α-hemoglobin remaining in solution. In FIG. 3c, oxy-β-hemoglobin was preincubated with AHSP, oxidized by ferricyanide and analyzed as described in FIG. 3a. FIG. 3d shows the absorption spectrum of oxygenated oxy-α-hemoglobin compared with oxy-α-hemoglobin preincubated with a 7.2-fold excess of AHSP, then treated with ferricyanide. The latter sample shows spectrophotometric changes consistent with oxidation of the heme-associated iron.

FIGS. 4a-4b show AHSP-α-hemoglobin interactions in mammalian cells. FIG. 4a is a blot depicting co-immunoprecipitates AHSP and α-hemoglobin expressed in COS cells. V5-epitope-tagged murine AHSP and hemagglutinin (HA)-tagged murine α-hemoglobin were expressed in COS cells and extracts were analyzed by immunoprecipitation (IP) followed by western blot analysis. FIG. 4b is a micrograph demonstrating that AHSP prevents α-hemoglobin precipitation in vivo. COS cells expressing HA-α-hemoglobin alone or in combination with V5-AHSP, were stained with antibodies against V5 and HA and analyzed by indirect immunofluorescence. Original magnification 400×.

FIGS. 5a-5d depict the strategy employed for targeted disruption of the AHSP gene. FIG. 5a shows a partial restriction map of the mouse AHSP locus (top) and the AHSP targeting vector (bottom). Gene exons are shown as black rectangles. The flanking probe used for Southern blot analysis is shown as a black bar. The targeting construct contains the HSV-tk and neo^{R} genes, both under the control of the phosphoglycerate kinase (PGK) promoter. Arrowheads indicate loxP sites to be used for removal of the neo^{R} gene. Homologous recombination results in removal of the entire coding sequence of AHSP, and introduces two PstI sites into the locus. P, PstI; X, XbaI; B, BlpI; Pc, PacI; S, SacI. Parentheses indicate loss of restriction sites during cloning. FIG. 5b shows a Southern blot of wild-type, and AHSP^{±} and AHSP^{-/-} gene-disrupted mice. Each lane represents 15 μg of genomic DNA digested with PstI. FIG. 5c shows a Northern blot analysis. Each lane represents 8 μg of total RNA. Blots performed in duplicate were probed with full-length cDNAs for either AHSP or globin. The AHSP blot was exposed approximately 20× longer than the globin blot. FIG. 5d shows a Western blot probed with antiserum prepared against recombinant AHSP protein. Each lane represents 10 μg of protein extract from spleen.

FIGS. 6a-6c show generation of EDRF-V5 transgenic mice (AHSP overexpressers.) FIG. 6a is a vector for transgenic expression of EDRF in murine erythroid progenitors. FIG. 6b shows southern blotting analysis of transgenic mouse lines. ˜15 μg Genomic DNA extracted from mouse tails was analyzed by Southern blotting with an EDRF cDNA probe. The endogenous (top) and transgenic (bottom) bands and their sizes are indicated on the left. Transgenic lines are indicated at the bottom. FIG. 6c shows V5 western blotting analysis of transgenic mouse lines. Total protein was extracted from adult mouse spleen and a V5 antibody was used as a probe.

FIGS. 7a-7b show AHSP-V5 transgene expression. FIG. 7a is a western blot of Magnetic Activated Cell Sorting (MACS) sorted bone marrow from transgenic mice showing erythroid-specific expression of the AHSP-V5 protein. FIG. 7b is a western blot of total bone marrow with an AHSP antibody, which shows that the AHSP-V5 transgene protein is expressed at similar levels to the endogenous AHSP protein.

FIGS. 8a-8d demonstrate that AHSP^{-/-} erythrocytes exhibit abnormal morphology, hemoglobin precipitates (Heinz bodies) and reduced lifespan. FIG. 8a shows blood hemoglobin concentrations expressed as means ±s.e.m. for mutant and wild-type littermates. No significant differences were observed in other erythrocyte indices including hematocrit, erythrocyte number, mean corpuscular volume, mean corpuscular hemoglobin and mean corpuscular hemoglobin concentration (not shown). FIG. 8b is a graph of blood reticulocyte counts expressed as percentages of total erythrocytes. Bars indicate means ±s.e.m. Single asterisk indicates P<0.05, double asterisk indicates P<0.01. FIG. 8c depicts Wright-Giemsa stains (upper panels) which show morphologic abnormalities and inclusion bodies (*) in mutant erythrocytes. Heinz body stain, which detects denatured globin chains (lower panels), is weakly positive in some AHSP^{±} erythrocytes (indicated by \hat{ }) and strongly positive in AHSP^{-/-} cells. Original magnification 400×. FIG. 8d depicts erythrocyte survival kinetics determined by biotin labeling. Circulating erythrocytes in 5 animals of each genotype were biotinylated at days −2 and −1. Approximately 5 μl of blood was removed from the tail vein at the indicated timepoints and the fraction of biotin-labeled erythrocytes was quantitated by flow cytometry. The half-life of wild-type red blood cells was 22 days, whereas that of the AHSP^{-/-} RBCs was 12 days.

FIGS. 9a-9e show erythroid hyperplasia and ineffective erythropoiesis in AHSP^{-/-} mice. FIG. 9a is a graph which depicts spleen weight in AHSP^{-/-} mice (n=6 animals for each genotype: *, p<0.005). FIG. 9b shows results from methylcellulose progenitor assays. CFUe: Colony Forming Unit-erythroid; BFUe: Bursting Forming Units-erythroid. (n=6 for each genotype). FIG. 9c depicts flow cytometry analysis of splenic erythroid precursors according to levels of Ter 119 and CD71 expression. Regions a through d represent increasingly mature stages of erythroid development (Socolovsky, M., et al. (2001) Blood, 98, 3261-3273) FIG. 9d shows elevated proportion of Ter119+ cells in AHSP^{-/-} mice (n=6 animals for each genotype, *:p<0.005). FIG. 9e shows the ratio of mature to immature erythroid precursors in spleen calculated according to levels of Ter 119 and CD 71 expression defined by flow cytometry gates in panel 9c: % mature cells=#cells in d/(a+b+c+d), n=6 animals for each genotype; **: p<0.05.

FIGS. 10a-10b depict unstable hemoglobins in AHSP^{-/-} erythrocytes. FIG. 10a shows Triton-Acetic Acid-Urea (TAU) gel analysis of membrane-associated globin chains with the AHSP genotypes indicated at the top. Each lane represents membrane skeletons prepared from the same number of erythrocytes. FIG. 10a depicts results from an isopropanol hemoglobin stability test. Isopropanol (17% v/v) was added to fresh hemolysates, incubated at 37° C. and hemoglobin precipitation was quantified. AHSP genotypes are indicated. The hemoglobin stabilities were significantly different at all timepoints for the two genotypes (n=5 animals of each genotype, p<0.01).

FIGS. 11a-c depict oxidative stress in AHSP^{-/-} erythrocytes. FIG. 11a shows relative ROS levels in erythrocytes measured by DCF fluorescence at baseline and with added H₂O₂. FIG. 11b shows protein oxidation in erythrocyte lysates. 20 μg of hemolysate was treated with 2,4-dinitrophenylhydrazine (DNPH) to derivatize carbonyl groups (by products of protein oxidation). Protein-associated DNP was then detected by Western blotting with anti-DNP antibody. An identical blot probed with anti-actin antibody (bottom) indicates equal protein loading in each lane. FIG. 11c depicts susceptibility to phenylhydrazine-induced hemolytic anemia. Drug was administered on days −1 and 0, then hematocrit (top panel) and reticulocyte count (bottom panel) were measured daily until recovery.

FIGS. 12a-12c show that AHSP prevents α Hb-mediated production of ROS and oxidation of Hb A in solution. FIG. 12a, is a graph showing results following incubation of α Hb or β Hb with AHSP on ice for 30 minutes followed by mixing with H₂O₂ and TMPD, a ROS indicator dye. The rate of TMPD oxidation, shown on the Y axis, was measured spectrophotometrically. The graph shows the average value of three experiments. FIG. 12b shows the effect of AHSP on H₂O₂-induced heme loss from α Hb in experiment from (12a), as measured by reduced absorbance at 412 nm. Molar ratio of a Hb:AHSP was 1:2. FIG. 12c shows the effect of AHSP or control protein SBTI on α Hb-mediated oxidation of Hb A. Hb A was incubated at 37° C. for 30 minutes and the extent of oxidation was measured spectrophotometrically. As indicated, α Hb was added to Hb A at a molar ratio of 1:8. To test the effects of AHSP, recombinant protein was preincubated with a Hb (1:1 molar ratio) for 30 minutes at 4° C. before adding to Hb A.

### DETAILED DESCRIPTION OF THE INVENTION

Alpha Hemoglobin Stabilizing Protein is an abundant, erythroid specific protein that forms a stable complex with free α-hemoglobin but not with β-hemoglobin or hemoglobin A (α-β). Moreover, AHSP specifically protects free α-hemoglobin from precipitation in solution and in live cells.

An abundance of α-hemoglobin presents a physiological danger to an organ system. Excess α-hemoglobin forms precipitates, called α-inclusion bodies, which damage red blood cells. Disclosed herein is the role of EDRF/AHSP as a molecular chaperone which binds α-hemoglobin, preventing precipitation and α-inclusion body formation. AHSP releases α-hemoglobin once it encounters β-hemoglobin, and thus plays a critical role in hemoglobin assembly. AHSP also appears to play a role in α-chain synthesis. Further, new data reveal that the AHSP transcript is down-regulated in mice having TSE. These discoveries provide a revolutionary step towards further diagnosis and treatment of a range of disorders in two broad categories—(1) anemias, particular those characterized by an excess of α-hemoglobin or a reduction of β-hemoglobin, and (2) for disorders characterized by formation of plaques or inclusion bodies, including neurological disorders, like TSE's and Alzheimer's disease.

I. Definitions

The term “Alpha Hemoglobin Stabilizing Protein” or “AHSP” refers to a 102 amino acid protein which is highly conserved in humans, pigs, cows, and rats. Genbank accession numbers for some AHSP molecules include Mus musculus, Accession No. AF485327, Bos tarus, Accession No. AF485326, Homo Sapien, Accession No. AF485325, Rattus Norvegicus, Accession No. XM_{—}215059, and Sus scrofa, Accession No. AW480086. ASHP is also sometimes referred to in the art as Erythroid Differentiation Related Factor (EDRF), or Erythroid Associated Factor (ERAF).

“AHSP related disorder” refers to any disorder which is characterized by mutation, deletion, or otherwise deleterious alterations of an AHSP molecule. Such disorders include but are not limited to α-thalassemia, β-thalassemia, anemia, transmissible spongiform encephalopathies (TSE's) such as bovine spongiform encephalopathy (BSE), Alzheimer's disease, and other disorders which are characterized by misassembly of hemoglobin, or the formation of plaques or inclusion bodies.

The term “nucleic acid molecule” describes a polymer of deoxyribonucleotides (DNA) or ribonucleotides (RNA). The nucleic acid molecule may be isolated from a natural source by cDNA cloning or subtractive hybridization or synthesized manually by the triester synthetic method or by an automated DNA synthesizer.

With regard to nucleic acids used in the invention, the term “isolated nucleic acid” is sometimes employed. This term, when applied to DNA, refers to a DNA molecule that is separated from sequences with which it is immediately contiguous (in the 5′ and 3′ directions) in the naturally occurring genome of the organism from which it was derived. For example, the “isolated nucleic acid” may comprise a DNA molecule inserted into a vector, such as a plasmid or virus vector, or integrated into the genomic DNA of a procaryote or eucaryote. An “isolated nucleic acid molecule” may also comprise a cDNA molecule. An isolated nucleic acid molecule inserted into a vector is also sometimes referred to herein as a recombinant nucleic acid molecule.

With respect to RNA molecules, the term “isolated nucleic acid” primarily refers to an RNA molecule encoded by an isolated DNA molecule as defined above. Alternatively, the term may refer to an RNA molecule that has been sufficiently separated from RNA molecules with which it would be associated in its natural state (i.e., in cells or tissues), such that it exists in a “substantially pure” form.

The term “complementary” describes two nucleotides that can form multiple favorable interactions with one another. For example, adenine is complementary to thymine as they can form two hydrogen bonds. Similarly, guanine and cytosine are complementary since they can form three hydrogen bonds. Thus if a nucleic acid sequence contains the following sequence of bases, thymine, adenine, guanine and cytosine, a “complement” of this nucleic acid molecule would be a molecule containing adenine in the place of thymine, thymine in the place of adenine, cytosine in the place of guanine, and guanine in the place of cytosine. Because the complement can contain a nucleic acid sequence that forms optimal interactions with the parent nucleic acid molecule, such a complement can bind with high affinity to its parent molecule.

With respect to single stranded nucleic acids, particularly oligonucleotides, the term “specifically hybridizing” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence. Appropriate conditions enabling specific hybridization of single stranded nucleic acid molecules of varying complementarity are well known in the art.

For instance, one common formula for calculating the stringency conditions required to achieve hybridization between nucleic acid molecules of a specified sequence homology is set forth below (Sambrook et al.,(1989) Molecular Cloning, Cold Spring Harbor Laboratory:
Tₘ=81.5° C.+16.6Log [Na+]+0.41(% G+C)−0.63 (% formamide)−600/#bp in duplex

As an illustration of the above formula, using [Na+]=[0.368] and 50% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C.

The stringency of the hybridization and wash depend primarily on the salt concentration and temperature of the solutions. In general, to maximize the rate of annealing of the probe with its target, the hybridization is usually carried out at salt and temperature conditions that are 20-25° C. below the calculated Tₘ of the hybrid. Wash conditions should be as stringent as possible for the degree of identity of the probe for the target. In general, wash conditions are selected to be approximately 12-20° C. below the Tₘ of the hybrid. In regards to the nucleic acids of the current invention, a moderate stringency hybridization is defined as hybridization in 6× SSC, 5× Denhardt's solution, 0.5% SDS and 100 μg/ml denatured salmon sperm DNA at 42° C., and washed in 2×SSC and 0.5% SDS at 55° C. for 15 minutes. A high stringency hybridization is defined as hybridization in 6×SSC, 5× Denhardt's solution, 0.5% SDS and 100 μg/ml denatured salmon sperm DNA at 42° C., and washed in 1×SSC and 0.5% SDS at 65° C. for 15 minutes. A very high stringency hybridization is defined as hybridization in 6×SSC, 5× Denhardt's solution, 0.5% SDS and 100 μg/ml denatured salmon sperm DNA at 42° C., and washed in 0.1×SSC and 0.5% SDS at 65° C. for 15 minutes.

The term “oligonucleotide,” as used herein refers to primers and probes of the present invention, and is defined as a nucleic acid molecule comprised of two or more ribo- or deoxyribonucleotides, preferably more than three. The exact size of the oligonucleotide will depend on various factors and on the particular application and use of the oligonucleotide. Oligonucleotides, which include probes and primers, can be any length from 3 nucleotides to the full length of the nucleic acid molecule, and explicitly include every possible number of contiguous nucleic acids from 3 through the full length of the polynucleotide. Preferably, oligonucleotides, which include probes and/or primers are at least about 10 nucleotides in length, more preferably at least 15 nucleotides in length, more preferably at least about 20 nucleotides in length.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and use of the method. For example, for diagnostic applications, depending on the complexity of the target sequence, the oligonucleotide probe typically contains 15-25 or more nucleotides, although it may contain fewer nucleotides. The probes herein are selected to be complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

Polymerase chain reaction (PCR) has been described in U.S. Pat. Nos. 4,683,195, 4,800,195, and 4,965,188, the entire disclosures of which are incorporated by reference herein.

The term “vector” relates to a single or double stranded circular nucleic acid molecule that can be transfected or transformed into cells and replicate independently or within the host cell genome. A circular double stranded nucleic acid molecule can be cut and thereby linearized upon treatment with restriction enzymes. An assortment of vectors, restriction enzymes, and the knowledge of the nucleotide sequences that are targeted by restriction enzymes are readily available to those skilled in the art, and include any replicon, such as a plasmid, cosmid, bacmid, phage or virus, to which another genetic sequence or element (either DNA or RNA) may be attached so as to bring about the replication of the attached sequence or element. A nucleic acid molecule of the invention can be inserted into a vector by cutting the vector with restriction enzymes and ligating the two pieces together.

Many techniques are available to those skilled in the art to facilitate transformation, transfection, or transduction of the expression construct into a prokaryotic or eukaryotic organism. The terms “transformation”, “transfection”, and “transduction” refer to methods of inserting a nucleic acid and/or expression construct into a cell or host organism. These methods involve a variety of techniques, such as treating the cells with high concentrations of salt, an electric field, or detergent, to render the host cell outer membrane or wall permeable to nucleic acid molecules of interest, microinjection, PEG-fusion, and the like. As used herein, the terms “reporter,” “reporter system”, “reporter gene,” or “reporter gene product” shall mean an operative genetic system in which a nucleic acid comprises a gene that encodes a product that when expressed produces a reporter signal that is a readily measurable, e.g., by biological assay, immunoassay, radio immunoassay, or by calorimetric, fluorogenic, chemiluminescent or other methods. The nucleic acid may be either RNA or DNA, linear or circular, single or double stranded, antisense or sense polarity, and is operatively linked to the necessary control elements for the expression of the reporter gene product. The required control elements will vary according to the nature of the reporter system and whether the reporter gene is in the form of DNA or RNA. Such elements may include, but are not be limited to, promoters, enhancers, translational control sequences, poly A addition signals, transcriptional termination signals and the like.

The introduced nucleic acid may or may not be integrated (covalently linked) into nucleic acid of the recipient cell or organism. In bacterial, yeast, plant and mammalian cells, for example, the introduced nucleic acid may be maintained as an episomal element or independent replicon such as a plasmid. Alternatively, the introduced nucleic acid may become integrated into the nucleic acid of the recipient cell or organism and be stably maintained in that cell or organism and further passed on or inherited to progeny cells or organisms of the recipient cell or organism. Finally, the introduced nucleic acid may exist in the recipient cell or host organism only transiently.

The term “selectable marker gene” refers to a gene that when expressed confers a selectable phenotype, such as antibiotic resistance, on a transformed cell.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of transcription units and other transcription control elements (e.g. enhancers) in an expression vector.

Amino acid residues described herein are preferred to be in the “L” isomeric form. However, residues in the “D” isomeric form may be substituted for any L-amino acid residue, provided the desired properties of the polypeptide are retained. All amino-acid residue sequences represented herein conform to the conventional left-to-right amino-terminus to carboxy-terminus orientation.

Amino acid residues are identified in the present application according to the three-letter or one-letter abbreviations in the following Table:

TABLE 1

| | 3-letter | 1-letter |
|---|---|---|
| Amino Acid | Abbreviation | Abbreviation |
| L-Alanine | Ala | A |
| L-Arginine | Arg | R |
| L-Asparagine | Asn | N |
| L-Aspartic Acid | Asp | D |
| L-Cysteine | Cys | C |
| L-Glutamine | Gln | Q |
| L-Glutamic Acid | Glu | E |
| Glycine | Gly | G |
| L-Histidine | His | H |
| L-Isoleucine | Ile | I |
| L-Leucine | Leu | L |
| L-Methionine | Met | M |
| L-Phenylalanine | Phe | F |
| L-Proline | Pro | P |
| L-Serine | Ser | S |
| L-Threonine | Thr | T |
| L-Tryptophan | Trp | W |
| L-Tyrosine | Tyr | Y |
| L-Valine | Val | V |
| L-Lysine | Lys | K |

The term “isolated protein” or “isolated and purified protein” is sometimes used herein. This term refers primarily to a protein produced by expression of an isolated nucleic acid molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity, and that may be present, for example, due to incomplete purification, addition of stabilizers, or compounding into, for example, immunogenic preparations or pharmaceutically acceptable preparations.

The term “biological activity” is a function or set of functions performed by a molecule in a biological context (i.e., in an organism or an in vitro surrogate or facsimile model). The biological activity of AHSP is preferably α-hemoglobin binding activity.

The term “tag,” “tag sequence” or “protein tag” refers to a chemical moiety, either a nucleotide, oligonucleotide, polynucleotide or an amino acid, peptide or protein or other chemical, that when added to another sequence, provides additional utility or confers useful properties, particularly in the detection or isolation, of that sequence. Thus, for example, a homopolymer nucleic acid sequence or a nucleic acid sequence complementary to a capture oligonucleotide may be added to a primer or probe sequence to facilitate the subsequent isolation of an extension product or hybridized product. In the case of protein tags, histidine residues (e.g., 4 to 8 consecutive histidine residues) may be added to either the amino- or carboxy-terminus of a protein to facilitate protein isolation by chelating metal chromatography. Alternatively, amino acid sequences, peptides, proteins or fusion partners representing epitopes or binding determinants reactive with specific antibody molecules or other molecules (e.g., flag epitope, c-myc epitope, transmembrane epitope of the influenza A virus hemaglutinin protein, protein A, cellulose binding domain, calmodulin binding protein, maltose binding protein, chitin binding domain, glutathione S-transferase, and the like) may be added to proteins to facilitate protein isolation by procedures such as affinity or immunoaffinity chromatography. Chemical tag moieties include such molecules as biotin, which may be added to either nucleic acids or proteins and facilitates isolation or detection by interaction with avidin reagents, and the like. Numerous other tag moieties are known to, and can be envisioned by the trained artisan, and are within the scope of this definition.

A “clone” or “clonal cell population” is a population of cells derived from a single cell or common ancestor by mitosis.

A “cell line” is a clone of a primary cell or cell population that is capable of stable growth in vitro for many generations.

An “antibody” or “antibody molecule” is any immunoglobulin, including antibodies and fragments thereof, that binds to a specific antigen. The term includes polyclonal, monoclonal, chimeric, and bispecific antibodies. As used herein, antibody or antibody molecule contemplates both an intact immunoglobulin molecule and an immunologically active portion of an immunoglobulin molecule such as those portions known in the art as Fab, Fab′, F(ab′)2 and F(v).

With respect to antibodies, the term “immunologically specific” refers to antibodies that bind to one or more epitopes of a protein or compound of interest, but which do not substantially recognize and bind other molecules in a sample containing a mixed population of antigenic biological molecules.

A “sample” or “patient sample” or “biological sample” generally refers to a sample which may be tested for a particular molecule, preferably AHSP polynucleotide, polypeptide, or antibody. Samples may include but are not limited to cells, including spleen cells, tissue, and body fluids, including blood, serum, plasma, cerebral-spinal fluid, urine, saliva, pleural fluid and the like.

A “patient” or “test subject” refers to an organism which may be tested for a particular disorder, including but not limited to a disorder characterized by overexpression of AHSP. A “patient” or “test subject” includes, but is not limited to animals, including mammalian species such as murine, porcine, ovine, bovine, canine, feline, equine, human, and other primates.

II. Transgenic Animals with an Altered AHSP Genotype

To further understand the role Alpha Hemoglobin Stabilizing Protein plays in disease processes, transgenic animals have been generated which comprise altered Alpha Hemoglobin Stabilizing Protein gene(s). The transgenic animals include homozygous and heterozygous AHSP deletion, as well as 2×AHSP expressing mice. The Alpha Hemoglobin Stabilizing Protein gene can be further altered to include modifications, deletions, and substitutions. Modifications and deletions render the naturally occurring gene nonfunctional, producing a “knock out” animal. Insertion of a transgene into fertilized oocytes produces increased gene copy number and expression. Substitutions of the naturally occurring gene for a gene from a second species results in an animal which produces an Alpha Hemoglobin Stabilizing Protein gene from the second species. Substitution of the naturally occurring gene for a gene having a mutation results in an animal with a mutated Alpha Hemoglobin Stabilizing Protein.

A transgenic animal carrying a “knock out” of Alpha Hemoglobin Stabilizing Protein is useful for the establishment of a nonhuman model for diseases involving under expression or non-expression of Alpha Hemoglobin Stabilizing Protein. A transgenic animal which overexpresses Alpha Hemoglobin Stabilizing Protein is useful for study of diseases pertaining to hemoglobin assembly. A transgenic mouse carrying the human Alpha Hemoglobin Stabilizing Protein gene is generated by direct replacement of the mouse Alpha Hemoglobin Stabilizing Protein gene with the human gene. These transgenic animals are useful as in vivo models for drug screening studies for human diseases, and for eventual treatment of disorders or diseases associated with aberrant Alpha Hemoglobin Stabilizing Protein.

As a means to define the role that AHSP plays in mammalian systems, mice have been generated that cannot make AHSP because of a targeted disruption of the AHSP gene. These mice develop normally and are fertile. However AHSP^{-/-} mice exhibit defective hemoglobin metabolism.

The phrase “transgenic animal” is used herein to include all vertebrate animals, except humans. It also includes an individual animal in all stages of development, including embryonic and fetal stages. A “transgenic animal” is any animal containing one or more cells bearing genetic information altered or received, directly or indirectly, by deliberate genetic manipulation at the subcellular level, such as by targeted recombination or microinjection or infection with recombinant virus. The term “transgenic animal” is not meant to encompass classical cross-breeding or in vitro fertilization, but rather is meant to encompass animals in which one or more cells are altered by or receive a recombinant DNA molecule. This molecule may be specifically targeted to a defined genetic locus, be randomly integrated within a chromosome, or it may be extrachromosomally replicating DNA. The term “germ cell line transgenic animal” refers to a transgenic animal in which the genetic alteration or genetic information was introduced into a germ line cell, thereby conferring the ability to transfer the genetic information to offspring. If such offspring possess some or all of that alteration or genetic information, then they, too, are transgenic animals.

The alteration or genetic information may be foreign to the species of animal to which the recipient belongs, or foreign only to the particular individual recipient, or may be genetic information already possessed by the recipient. In the last case, the altered or introduced gene may be expressed differently than the native gene.

The altered Alpha Hemoglobin Stabilizing Protein gene generally should not fully encode the same Alpha Hemoglobin Stabilizing Protein native to the host animal and its expression product should be altered to a minor or great degree, or absent altogether. However, it is conceivable that a more modestly modified Alpha Hemoglobin Stabilizing Protein gene will fall within the scope of the present invention if it is a specific alteration.

The DNA used for altering a target gene may be obtained by a wide variety of techniques that include, but are not limited to, isolation from genomic sources, preparation of cDNAs from isolated mRNA templates, direct synthesis, or a combination thereof.

A type of target cell for transgene introduction is the embryonal stem cell (ES). ES cells may be obtained from pre-implantation embryos cultured in vitro.(Evans et al., (1981) Nature 292:154-156; Bradley et al., (1984) Nature 309:255-258; Gossler et al., (1986) Proc. Natl. Acad. Sci. 83:9065-9069) Transgenes can be efficiently introduced into the ES cells by standard techniques such as DNA transfection or by retrovirus-mediated transduction. The resultant transformed ES cells can thereafter be combined with blastocysts from a non-human animal. The introduced ES cells thereafter colonize the embryo and contribute to the germ line of the resulting chimeric animal.

One approach to the problem of determining the contributions of individual genes and their expression products is to use isolated Alpha Hemoglobin Stabilizing Protein (AHSP) genes to selectively inactivate the wild-type gene in totipotent ES cells (such as those described above) and then generate transgenic mice. The use of gene-targeted ES cells in the generation of gene-targeted transgenic mice has been described, and is reviewed elsewhere (Frohman et al., (1989) Cell 56:145-147; Bradley et al., (1992) Bio/Technology 10:534-539)

Techniques are available to inactivate or alter any genetic region to a mutation desired by using targeted homologous recombination to insert specific changes into chromosomal alleles. However, in comparison with homologous extrachromosomal recombination, which occurs at a frequency approaching 100%, homologous plasmid-chromosome recombination was originally reported to only be detected at frequencies between 10⁻⁶ and 10⁻³. Nonhomologous plasmid-chromosome interactions are more frequent, occurring at levels 10⁵-fold to 10²-fold greater than comparable homologous insertion.

To overcome this low proportion of targeted recombination in murine ES cells, various strategies have been developed to detect or select rare homologous recombinants. One approach for detecting homologous alteration events uses the polymerase chain reaction (PCR) to screen pools of transformant cells for homologous insertion, followed by screening of individual clones. Alternatively, a positive genetic selection approach has been developed in which a marker gene is constructed which will only be active if homologous insertion occurs, allowing these recombinants to be selected directly. One of the most powerful approaches developed for selecting homologous recombinants is the positive-negative selection (PNS) method developed for genes for which no direct selection of the alteration exists. The PNS method is more efficient for targeting genes which are not expressed at high levels because the marker gene has its own promoter. Non-homologous recombinants are selected against by using the Herpes Simplex virus thymidine kinase (HSV-TK) gene and selecting against its nonhomologous insertion with effective herpes drugs such as gancyclovir (GANC) or (1-(2-deoxy-2-fluoro-B-D arabinofluranosyl)-5-iodouracil, (FIAU). By this counter selection, the fraction of homologous recombinants in the surviving transformants can be increased.

As used herein, a “targeted gene” or “knock-out” is a DNA sequence introduced into the germline of a non-human animal by way of human intervention, including but not limited to, the methods described herein. The targeted genes of the invention include DNA sequences which are designed to specifically alter cognate endogenous alleles.

Methods of use for the transgenic mice of the invention are also provided herein. Such mice may be used to identify agents which augment, inhibit, or modify the activities of Alpha Hemoglobin Stabilizing Protein (AHSP). For example, therapeutic agents for the treatment or prevention of anemia may be screened in studies using Alpha Hemoglobin Stabilizing Protein (AHSP) knock out mice. In one embodiment, a compound or combination of compounds are administered to an AHSP^{-/-} mouse having abnormal erythrocyte morphology and/or increased reticulocyte count, and then samples from the mouse are analyzed to determine if there has been an improvement in the mouse's erythrocyte morphology and/or reticulocyte count. In another embodiment, AHSP knockout mice may be treated with a test compound that induces anemia. Secondary reagents could then be assessed which inhibit or suppress the anemia pathway.

Accordingly, it has previously been determined that AHSP levels are dramatically reduced in mice having transmissible spongiform encephaopathies or prion disease. The mice of the invention may also be used to study the progression of prion disease, to further elucidate the role of AHSP in prion disease, and/or to test agents for efficacy in preventing or treating prion disease. In one embodiment, AHSP null mice, or mice which overexpress AHSP are infected with an agent that causes a spongiform encephalopathy (prion disease) as previously described (Miele, G. et al. (2001) Nature Medicine 7:361-364), and these mice are monitored to measure disease progression. In another embodiment, mice of the invention may be administered an agent, thereby determining the effect of that agent on the progression of prion disease. Mice may be infected with the agent that causes prion disease by methods known in the art, such as by intracerebral inoculation (Thackray et al., (2002) Journal of Virology, Vol. 76. No. 5, p. 2510-2517). Alternatively, primary tissue culture from the mice of the invention may be infected with prion disease using a transfection vector (Parizek et al., (2001) The Journal of Biological Chemistry, Vol. 246, No. 48, pp 44627-44632).

Such assays will not only facilitate the identification of agents which cause or inhibit anemia and/or prion disease, they should also be illustrative of the biochemical mechanisms which underlie these disorders.

III. Methods of Using AHSP Polynucleotides, Polypeptides, and Antibodies for Screening and Diagnostic Assays

In accordance with the link between hemoglobin assembly and AHSP disclosed herein, numerous assays may be utilized to test for pathologies ranging from anemia to neurological disorders. Further, additional studies may be conducted to identify other disorders associated with aberrant AHSP expression.

AHSP-encoding DNA, RNA, or fragments thereof may be used as probes to detect the presence of and/or expression of genes encoding AHSP proteins. Methods in which AHSP-encoding nucleic acids may be utilized as probes for such assays include, but are not limited to: (1) in situ hybridization; (2) Southern hybridization (3) northern hybridization; and (4) assorted amplification reactions such as polymerase chain reactions (PCR).

Polyclonal or monoclonal antibodies immunologically specific for AHSP may be used in a variety of assays designed to detect and quantitate the protein. Such assays include, but are not limited to: (1) flow cytometric analysis; (2) immunochemical localization of AHSP in blood or bone marrow; and (3) immunoblot analysis (e.g., dot blot, Western blot) of extracts from blood or bone marrow. Additionally, anti-AHSP antibodies can be used for purification of AHSP (e.g., affinity column purification, immunoprecipitation).

From the foregoing discussion, it can be seen that AHSP-encoding nucleic acids, AHSP expressing vectors, AHSP proteins and anti-AHSP antibodies of the invention can be used to detect AHSP gene expression, and to alter AHSP expression. For example, these molecules can be used to detect AHSP gene expression in body fluids including blood, bone marrow, and/or spleen cells. Additionally, these molecules can be used to alter AHSP protein expression for purposes of assessing the genetic and protein interactions involved in hemoglobin assembly, anemia, and neurodegenerative disorders.

Exemplary approaches for detecting AHSP nucleic acid or polypeptides/proteins include:
a) comparing the sequence of nucleic acid in the sample with the AHSP nucleic acid sequence to determine whether the sample from the patient contains mutations; or
b) determining the presence, in a sample from a patient, of the polypeptide encoded by the AHSP gene and, if present, determining whether the polypeptide is full length, and/or is mutated, and/or is expressed at the normal level; or
c) using DNA restriction mapping to compare the restriction pattern produced when a restriction enzyme cuts a sample of nucleic acid from the patient with the restriction pattern obtained from normal AHSP gene or from known mutations thereof; or,
d) using a specific binding member capable of binding to a AHSP nucleic acid sequence (either normal sequence or known mutated sequence), the specific binding member comprising nucleic acid hybridizable with the AHSP sequence, or substances comprising an antibody domain with specificity for a native or mutated AHSP nucleic acid sequence or the polypeptide encoded by it, the specific binding member being labeled so that binding of the specific binding member to its binding partner is detectable; or,
e) using PCR involving one or more primers based on normal or mutated AHSP gene sequence to screen for normal or mutant AHSP gene in a sample from a patient.

A “specific binding pair” comprises a specific binding member (sbm) and a binding partner (bp) which have a particular specificity for each other and which in normal conditions bind to each other in preference to other molecules. Examples of specific binding pairs are antigens and antibodies, ligands and receptors and complementary nucleotide sequences. The skilled person is aware of many other examples. Further, the term “specific binding pair” is also applicable where either or both of the specific binding member and the binding partner comprise a part of a large molecule. In embodiments in which the specific binding pair are nucleic acid sequences, they will be of a length to hybridize to each other under conditions of the assay, preferably greater than 10 nucleotides long, more preferably greater than 15 or 20 nucleotides long.

In most embodiments for screening for AHSP expression associated with hemoglobin assembly, and anemia or neurodegenerative disorders, the AHSP nucleic acid in the sample will initially be amplified, e.g. using PCR, to increase the amount of the template as compared to other sequences present in the sample. This allows the target sequences to be detected with a high degree of sensitivity if they are present in the sample. This initial step may be avoided by using highly sensitive array techniques that are becoming increasingly important in the art.

Decreased expression levels of Alpha Hemoglobin Stabilizing Protein (AHSP) have been linked to disorders such as β-thalassemia, and spongiform encephalopathies. Accordingly, tests which detect AHSP expression levels in patients suspected of having a disorder such as β-thalassemia, and spongiform encephalopathy could provide helpful diagnostic information, if a decrease in AHSP expression is detected in comparison to control patients.

Thus any of the aforementioned techniques may be used as a diagnostic tool for detecting decreased AHSP expression associated with anemias (such as β-thalassemia) or neurodegenerative disorders (such as spongiform encephalopathies), as well as for identifying other disorders which differentially express AHSP. These techniques are generally known in the art, and more specific protocols can be found in materials such as Maniatis et al., (1989) Molecular Cloning: A Laboratory Manual, Cold Spring Harbor Laboratory Press, NY, Vol. 1, 2, 3.

IV. Methods of Using AHSP Polynucleotides for Antisense and Gene Therapy

Nucleic acid molecules, or fragments thereof, encoding AHSP may also be utilized to enhance the production of AHSP, thereby increasing the amount of protein available to participate in hemoglobin stabilization. Alternatively, reverse orientation polynucleotides may be used to inhibit expression of AHSP polypeptide. Alterations in the physiological amount of AHSP protein may dramatically affect the levels of precipitated α-hemoglobin.

Ideally, vectors such as viral vectors, can be used to introduce AHSP encoding nucleic acid constructs into a wide variety of different target cells. Typically the vectors are exposed to the target cells so that transformation can take place in a sufficient proportion of the cells to provide a useful therapeutic or prophylactic effect from the expression of the desired polypeptide. The transfected nucleic acid may be permanently incorporated into the genome of each of the targeted cells, providing long lasting effect, or alternatively the treatment may have to be repeated periodically.

A variety of vectors, both viral vectors and plasmid vectors are known in the art, see U.S. Pat. No. 5,252,479 and WO 93/07282. In particular, a number of viruses have been used as gene transfer vectors, including papovaviruses, such as SV40, vaccinia virus, herpes viruses including HSV and EBV, and retroviruses.

Alternatively, should it be desirable to inhibit AHSP expression, vectors encoding a toxic protein operably linked to the AHSP promoter may be utilized. Expression of the toxic protein triggers cell death in the recipient cells. Also, antisense nucleic acid molecules may be targeted to translation initiation sites and/or splice sites to inhibit the expression of the AHSP genes or production of their encoded proteins. Antisense oligonucleotides may be designed to hybridize to the complementary sequence of nucleic acid, pre-mRNA or mature mRNA, interfering with the production of polypeptides encoded by a given DNA sequence (e.g. either native AHSP polypeptide or a mutant form thereof), so that its expression is reduced or prevented altogether. In addition to the AHSP coding sequence, antisense techniques can be used to target control sequences of the AHSP gene, e.g. in the 5′ flanking sequence of the AHSP coding sequence, whereby the antisense oligonucleotides can interfere with AHSP control sequences. The construction of antisense sequences and their use is described in Peyman and Ulman, (1990) Chemical Reviews, 90:543-584; Crooke, (1992) Ann. Rev. Pharmacol. Toxical., 32:329-376; and Zamecnik and Stephenson, (1974) Proc. Natl. Acad. Sci., 75:280-284. Such antisense molecules are typically between 15 and 30 nucleotides in length and often span the translational start site of the AHSP mRNA molecules.

Alternatively, antisense constructs may be generated which contain the entire AHSP cDNA in reverse orientation.

V. Kits for Detecting AHSP and Diagnosing AHSP Related Disorders

Any of the aforementioned AHSP based products or methods can be incorporated into a kit which may contain one or more polynucleotide, oligonucleotide, polypeptide, peptide, antibody, label, marker, or reporter, pharmaceutically acceptable carrier, physiologically acceptable carrier, instructions for use, container, vessel for administration, assay substrate, or any combination thereof.

### EXAMPLES

The following examples are provided to illustrate certain embodiments of the invention. They are not intended to limit the invention in any way.

### Example I

### Determination of AHSP's Role in Hemoglobin Binding

The following example demonstrates that EDRF/AHSP functions as a hemoglobin stabilizer. The materials and methods set forth below are provided to facilitate the practice of Example I.

Cell Lines

G1E-ER2 cells were cultured and induced to differentiate with β-oestradiol as described. MEL cells were grown in Dulbecco's modified Eagle's medium (Gibco-BRL)supplemented with 10% fetal bovine serum. To induce erythroid maturation, cells were seeded at a density of 10⁵ ml in medium containing 2% dimethyl sulphoxide (DMSO).

Plasmids

Human and mouse AHSP cDNAs were isolated by RT-PCR from fetal liver with SuperScriptII reverse transcriptase (Gibco-BRL) and Pfu polymerase (Stratagene). PCR primers were as follows: mouse AHSP, 5′-GGATGAGGCGGGCTCAGCACCATTA-3′ (SEQ ID NO:1) and 5′-TCGGCTCACAAACCCCAAGATTC-3′ (SEQ ID NO:2); human AHSP, 5′-TGAAGGCAGATGGCTCTTC-3′ (SEQ ID NO:3) and 5′-GTCCCTGAGCTAGGAGGAGG-3′ (SEQ ID NO:4). PCR conditions were 94° C. for 30 s, 55° C. for 30 s, 72° C. for 45 s (30 cycles). cDNAs derived by RT-PCR were sequenced on both strands.

Proteins

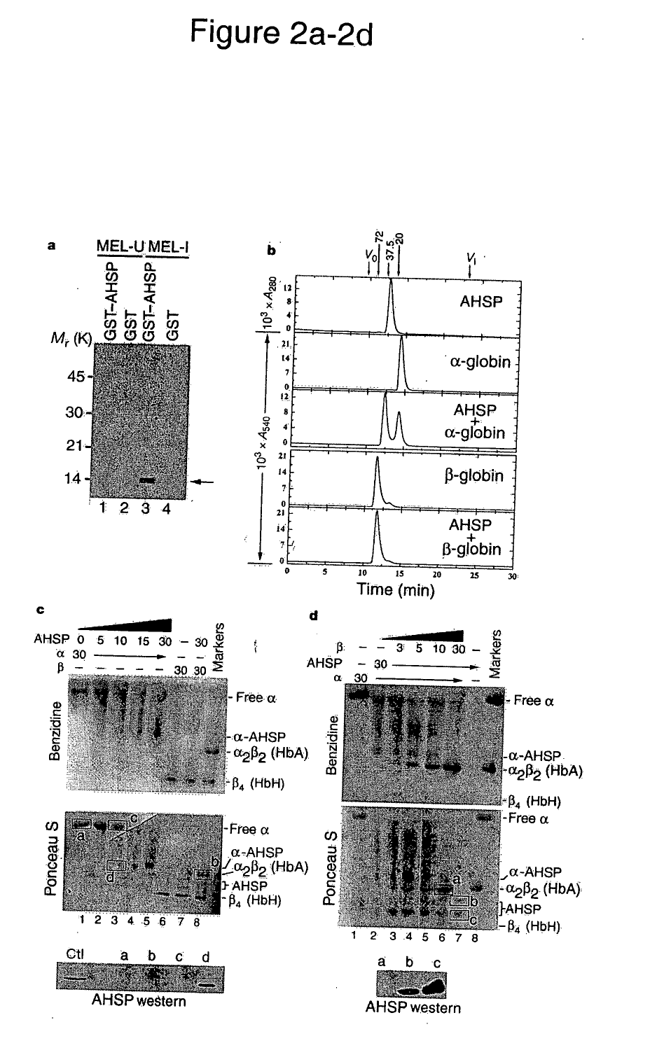

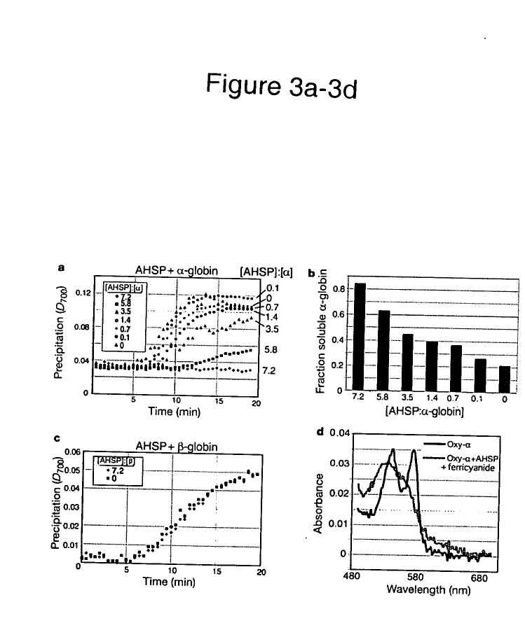

GST-fused mouse and human AHSP were prepared with the vector pGEX 6P-1(Amersham-Pharmacia) in accordance with the manufacturer's instructions. Human hemoglobins were prepared as described. Carboxyhemoglobins were used for protein interaction studies (FIG. 2b-d) because this enhances protein stability by limiting the generation of oxidative products. Oxy-α- and oxy-β-hemoglobins were used for studies on protein stability (FIG. 3).

Western Blot Analysis

Western blotting was performed using Hybond C extra (Amersham-Pharmacia), according to the manufacturer's instructions. Bound antibody was detected using Supersignal West Pico (Pierce). Rat anti-mouse AHSP antibody was prepared by immunizing rats with affinity purified GST-murine AHSP.

Northern Blot Analysis

RNA was isolated from various tissues and cell lines using Trizol (Gibco-BRL), fractionated on 1.2% agarose-formaldehyde gels, transferred to Hybond C+ and hybridized with a ³²P-labeled full-length AHSP cDNA probe. Blots were washed at a final stringency of 0.5×SSC at 65° C.

Analysis of AHSP Expression in Hematopoietic Colonies

Hematopoietic colonies were generated by in vitro differentiation of wild type or GATA-1− ES cells. Cellular mRNA was amplified by a modification of the poly(A)+ PCR method (Brady, G. & Iscove, N. Methods Enzymol 225, 611-623 (1993), Shirihai, et al., EMBO J. 19, 2492-2502 (2000)). The amplified products were denatured and transferred to Hybond N+ membrane (Amersham-Pharmacia) using a “slot blot” apparatus. Membranes were hybridized with ³²P-labeled cDNA fragments encoding AHSP or the constitutively expressed ribosomal protein L32.

Yeast Two-hybrid Screen

The Matchmaker GAL4 two-hybrid system (Clontech) was used. Full length E1 was used as bait to screen a MEL cell cDNA library (Tsang, et al.(1997) Cell 90, 109-119). The yeast strain made use of triple selection to minimize isolation of false positive clones. True positive clones were defined as those that induced the expression of 3 independent selectable markers (lacZ, ADE-2 and HIS-3). To rule out false positives due to yeast mutations that result in non-specific activation of a reporter gene, isolated plasmid clones (prey) were retransformed into yeast strains expressing bait plasmid alone, or control plasmid lacking the bait.

GST-pulldown

Recombinant GST fusion proteins were cross-linked to agarose beads (Harlow, E. & Lane, D. Using Antibodies: A Laboratory Manual, (1998) Cold Spring Harbor Laboratory Press, Cold Spring Harbor, N.Y.). MEL cells were lysed by incubating for 10 minutes at 4° C. in hypertonic lysis buffer [420 mM NaCl, 50 mM Tris pH 7.4, 0.5% IPEGAL, 1 mM dithiothreitol and protease inhibitor cocktail (Sigma)]. The lysates were cleared by centrifugation and the NaCl concentration adjusted to 150 mM by addition of lysis buffer without NaCl. One hundred μg cell lysate protein was added to 25 μg AHSP-GST or GST alone, cross-linked to agarose beads, and incubated with gentle rocking for 4 hours at 4° C. The beads were washed 3 times with lysis buffer containing 150 mM NaCl. Bound protein was removed by boiling in Laemmli sample buffer and analyzed by 12.5% SDS polyacrylamide gel electrophoresis followed by staining with Coomassie blue. The visible protein band was excised from the gel, eluted and digested with trypsin. The tryptic fragments were separated by anion exchange chromatography and sequenced by Edman degradation (Reim, et al. (1997) Current Protocols in Protein Science 11.10.11-38 John Wiley and Sons Inc., New York).

Immunoprecipitation

COS cells were transfected with cDNA expression vectors encoding V5 epitope-tagged AHSP and HA-tagged α-hemoglobin using FUGENE reagent (Roche). Cells were harvested, washed with phosphate buffered saline, and incubated for 10 minutes at 4° C. in hypertonic lysis buffer. Lysates were cleared by centrifugation and adjusted to 150 mM NaCl. Eight μg mouse monoclonal antibodies, either anti-HA, 0.2 μg/ml, (Roche) or anti-V5, 0.2 μg/ml, (InVitrogen), or control mouse IgG were added, along with 30 μl protein A agarose beads (50% slurry, Pierce) and the samples incubated for 4 hours at 4° C. Beads were washed extensively as described above. Bound protein was analyzed by western blotting.

Size Exclusion Chromatography

Gel filtration was performed using a TSK-GEL column (G2000SWXL, Tosohaas) on a Biocad Sprint Perfusion Chromatography System (PerSeptive Biosystems). The mobile phase was 50 mM Hepes pH 7.4, 150 mM NaCl, 1 mM EDTA. Flow rate was 0.75 ml/min.

Hemoglobin Precipitation Studies

Human hemoglobins were diluted with 50 mM Tris pH 7.4, 150 mM NaCl, 1 mM EDTA to a final concentration of 13.5 μM and preincubated at 4° C. with recombinant AHSP or control proteins in a volume of 90 μl for 60 minutes. The samples were warmed to 37° C. and 10 μl of potassium ferricyanide 0.5 mM was added. Protein precipitation was quantified by light scattering at 700 nm, using a Spectramax 250 plate reader (Molecular Devices). Spectral analysis of hemoglobin proteins was performed using the same instrument.

Immunofluorescence

AHSP-V5 and a hemoglobin-HA were cloned into the vector pEF1α and transfected into COS cells using Fugene (Roche). In some experiments 30 μM hemin was added as a source of exogenous heme, although results were similar when hemin was omitted. Samples were fixed with paraformaldehyde and incubated with rabbit-anti V5 (InVitrogen 1 μg/ml) and mouse-anti-HA (Santa Cruz, 1 μg/ml). Bound antibodies were detected with FITC labeled anti-rabbit IgG (Jackson Immunoresearch, 2 μg/ml) and Texas Red labeled anti-mouse IgG (Jackson Immunoresearch 10 μg/ml). Immunolabeled cultures were sectioned optically, using a computer-interfaced, laser-scanning microscope (Leica TCS 4D).

Results/Discussion

Identification of interacting partners which interact with AHSP was undertaken to further elucidate AHSP function. Full-length AHSP was used as bait to screen a murine erythroleukemia (MEL) cell complementary DNA library in a yeast two-hybrid assay. Analysis of 7.4×10⁵ colonies yielded 37 positive clones, each encoding α-globin protein. As an independent approach, it was demonstrated that a glutathione S-transferase (GST)-AHSP fusion protein selectively bound free α-hemoglobin in MEL cell extracts (FIG. 2a). To determine whether AHSP and hemoglobins interact in solution, recombinant human AHSP was incubated with α- or β-hemoglobins and the reactions were analyzed by gel-filtration chromatography (FIG. 2b). Eluted hemoglobins were detected by light absorbance at 540 nm, a property conferred by the heme prosthetic group. AHSP was monitored by absorbance at 280 nm. Although pure α-hemoglobin exists primarily as a monomer in solution, incubation with AHSP generated a larger heme-protein species (FIG. 2b) that contained both α-hemoglobin and AHSP by western blotting of collected fractions (not shown). Gel-filtration studies, using various concentrations of AHSP and α-hemoglobin to estimate binding equilibrium, indicated a maximum dissociation constant (Kd) of 0.5 mM (not shown). Pure β-hemoglobin exists mainly as a tetramer (β4, HbH), which did not interact with AHSP in solution (FIG. 2b, bottom two panels).

The specificity of AHSβ-hemoglobin interactions were studied by incubating purified hemoglobins with each other, or with recombinant AHSP, and resolving the complexes by non-denaturing cellulose acetate electrophoresis. Samples analyzed in parallel were stained with either benzidine, which detects heme-containing proteins, or Ponceau S, which detects all proteins (FIGS. 2c, d). Free α-hemoglobin migrated slowly and stained with both reagents (FIG. 2c, lane 1), whereas AHSP migrated as a fast-moving doublet that stained only with Ponceau S (FIG. 2d, lane 7). Incubation of AHSP with α-hemoglobin resulted in the formation of an α-hemoglobin-AHSP complex (FIG. 2c, lanes 2-5) that stained with benzidine and contained AHSP, as determined by western blotting of protein extracted from the cellulose acetate matrix. AHSP did not affect the mobility of hemoglobin H (FIG. 2c, lane 7) or hemoglobin A (not shown). Taken together, the gel-filtration and cellulose acetate electrophoretic studies demonstrate that AHSP forms a specific and stable complex with α-hemoglobin.

The α-hemoglobin-AHSP complex was disrupted by the addition of β-hemoglobin, with the concomitant appearance of hemoglobin A (α₂β₂), which did not contain AHSP as detected by western blotting (FIG. 2d, lanes 3-6). Hence, AHSP and β-hemoglobin compete for α-hemoglobin binding, with β-hemoglobin binding more tightly.

Next, it was investigated whether AHSP alters functionally important physico-chemical properties of α-hemoglobin. Free α-hemoglobin is highly unstable in cells, forming inclusion bodies that are detected by staining with crystal violet (Heinz bodies). These inclusion bodies decrease erythrocyte lifespan by damaging and destabilizing the cell membrane(Nathan et al., (1966) Am. J. Med, Vol. 41, pages 815-830; Nathan et al., (1990) J. Clin. Invest, Vol. 48, pages 33-41; Shinar et al., (1987) Blood, Vol. 70, pages 158-164; Sorensen et al., (1990) Blood, Vol. 75, pages 1333-1336; Yuan et al., (1995) Blood, Vol. 82, pages 374-377.) Precipitated α-hemoglobin can also cause apoptosis of erythroid precursors in the bone marrow and spleen (Rachmilewitz et al., (2001) Disorders in Hemoglobin, page 233-251, Cambridge University Press, Cambridge; Yuan et al., (1993) Blood, Vol. 82, pages 374-377; Pootrakul et al., (2000) Blood, Vol. 96, pages 2606-2612; Centis et al., (2000) Blood, Vol. 96, pages 3624-3629.) These events underlie the pathophysiology of ineffective erythropoiesis in β-thalassemia, in which defects in β-hemoglobin production lead to an excess of α-subunits and the formation of inclusion bodies. The precipitation of α-hemoglobin in vivo is triggered by oxidation of heme-bound iron, initiating a series of structural perturbations that culminate in protein denaturation.

This molecular cascade is modeled in vitro by exposing free α-hemoglobin to the oxidant ferricyanide, which induces its rapid precipitation (FIG. 3a)(Rachmilewitz et al., (1971) J. Biol. Chem. 246, pages 3356-3366.) Remarkably, AHSP, but not the control proteins IgG or GST, blocked oxidant-induced α-hemoglobin precipitation in a dose-dependent fashion (FIGS. 3a, b, and not shown). In contrast, oxidant-induced precipitation of β-hemoglobin (FIG. 3c) was not inhibited by AHSP, indicating that its stabilizing effects are specific for hemoglobin subunits. Spectrophotometric analysis demonstrated that AHSP-bound α-hemoglobin was oxidized by ferricyanide (FIG. 3d). AHSP therefore prevents the precipitation of oxidized α-hemoglobin rather than simply acting as an anti-oxidant.

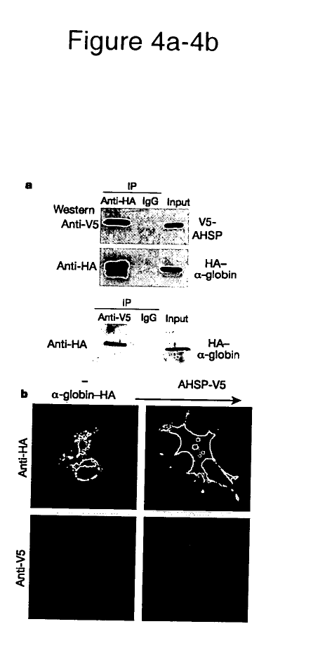

To determine whether AHSP and α-hemoglobin interact in vivo, epitope-tagged versions of each protein were expressed in COS cells (FIG. 4). Coexpressed AHSP and α-hemoglobin immunoprecipitated together from cell extracts, which is consistent with complex formation in vivo (FIG. 4a). Protein distribution was then detected by indirect immunofluorescence with the use of laser confocal microscopy (FIG. 4b). When expressed alone, α-hemoglobin precipitated in a punctate pattern, in a similar manner to inclusion body formation in β-thalassemic erythroid precursors that contain excess free α-hemoglobin (Wickramasinghe et al., (1980) Br. J. Hematol., Vol. 46, pages 401-407.) In contrast, α-hemoglobin was distributed homogeneously throughout the cytoplasm when expressed with AHSP, showing that AHSP can prevent α-hemoglobin precipitation in intact cells.

Most cells use two general strategies for neutralizing unstable proteins: proteolysis and stabilization by molecular chaperones(Wickner et al., (1999) Science, Vol. 286, pages 1888-1893.) Proteolytic pathways exist that degrade free α-hemoglobin in erythroid precursors (Bank et al., (1969) Nature Vol. 222, pages 295-296; Shaeffer et al., (1983) J. Biol. Chem, Vol. 258, pages 13172-13177.) Direct evidence is provided for an erythroid-specific molecular chaperone for free α-hemoglobin. These two complementary mechanisms, acting independently or coordinately, help explain how individuals with the β-thalassemia trait tolerate a deficit in β-hemoglobin production with no α-hemoglobin precipitation in erythroid precursors(Huang et al., (2001) Disorders of Haemoglobin, 146-173, Cambridge University Press, Cambridge.) The disclosed model predicts that full or partial loss of AHSP function would exacerbate mild or intermediate forms of β-thalassemia. Conversely, delivery of AHSP or functional analogues are predicted to decrease ineffective erythropoiesis caused by the precipitation of α-hemoglobin in β-thalassemia.

### Example II

### Generation of AHSP Transgenic Mice

For further study and characterization of the role AHSP plays in hemoglobin assembly and associated disorders, AHSP transgenic mice which express reduced levels or no AHSP, and mice which overexpress AHSP are provided.

Targeted Disruption of the AHSP Gene

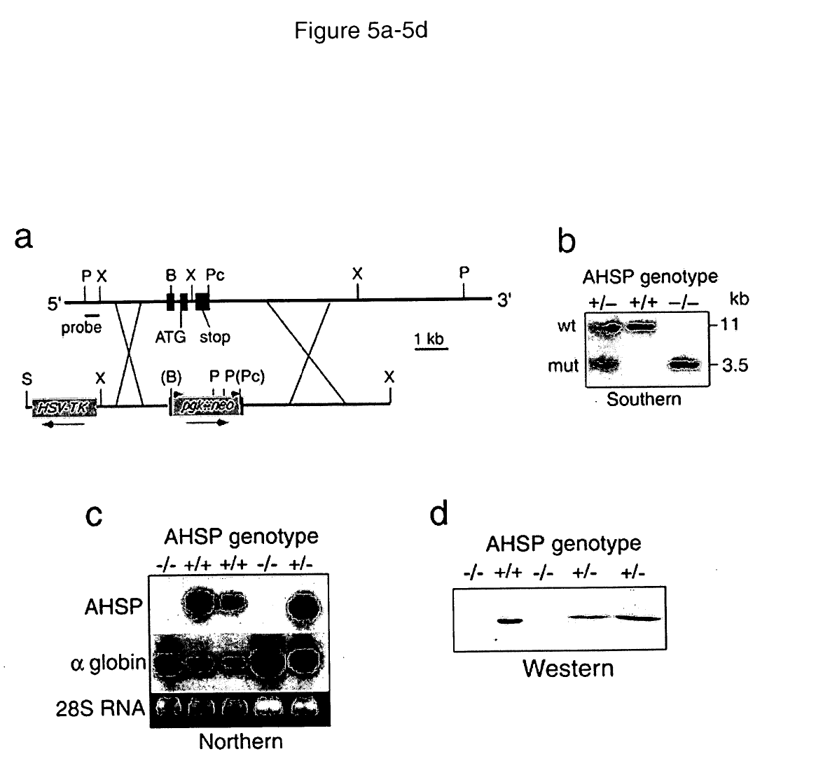

A mouse bacterial artificial chromosome (BAC) library (Research Genetics) was screened by PCR using AHSP gene primers. A positive BAC was isolated and an 11 kb PstI plasmid subclone containing the entire AHSP gene was purified and used for construction of the targeting vector. The 1 kb BlpI-PacI fragment containing the entire coding sequence was removed and replaced with floxed pgk-neo. HSK-thymidine kinase was ligated to the XbaI site. The AHSP targeting plasmid was linearized with SacI and electroporated into ES cells (strain 129, obtained from Trish Labosky; also available from Research Genetics, Invitrogen, Carlsbad, Calif.) Clones with stable integration of the targeting vector were selected in G418 and gancyclovir. Clones in which a single allele of AHSP was deleted by homologous recombination were identified by Southern blotting according to the strategy outlined in FIG. 5. One AHSP gene-deleted heterozygous clone with a normal karyotype was injected into blastocysts to produce chimeric mice. These were bred with wild-type mice (strain C57BL6) to produce AHSP^{±} heterozygotes, which were interbred to produce AHSP^{-/-} homozygotes. Southern, Northern and Western blots analyses of mouse tissues were performed according to standard methods.

Hematologic Analysis

Experiments were carried out under protocols approved by the Institutional Animal Care and Use Committee at The Children's Hospital of Philadelphia. Blood was collected by retroorbital puncture of anesthetized mice at 5-6 weeks age. Complete blood counts were determined using a Hemavet 850 (CDC technologies). Blood smears were prepared by standard methods. Reticulocytes were identified by methylene blue staining of blood smears and enumerated by three independent observers who were blinded to AHSP genotype. Heinz bodies were visualized by staining erythrocytes with crystal violet. Hemoglobin cellulose acetate electrophoresis was performed on chromatography strips (Helena Labs) according to the manufacturer's instructions.

Generation of AHSP-overexpressing Mice

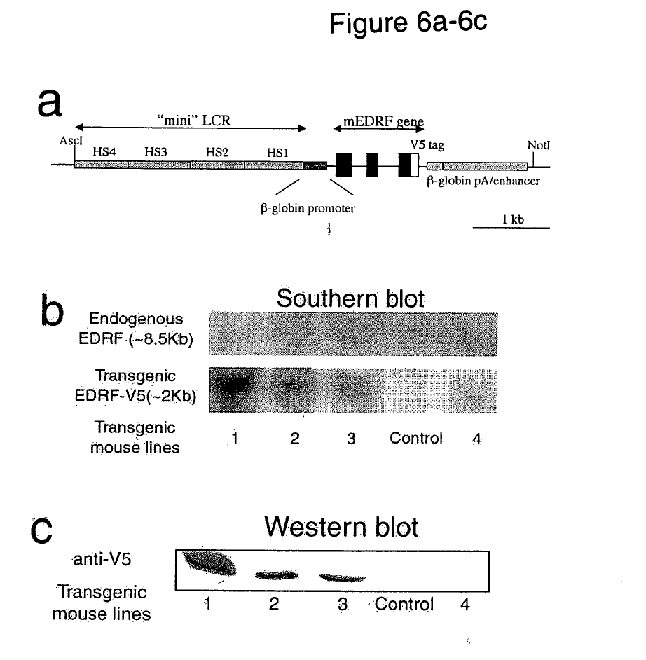

A transgenic vector was designed in which AHSP expression is driven by sequences from the promoter and mini locus control region (LCR) of the human beta globin gene (FIG. 6a). These regulatory elements have previously been demonstrated to give rise to high levels of erythroid-specific expression in an integration site-independent, copy number-dependent fashion. A V5 epitope tag was added to the carboxyl-terminus of AHSP to facilitate analysis of the transgene protein. Control experiments established that the V5 tag does not interfere with the ability of AHSP to stabilize free alpha globin. Linearized transgenic vector was injected into fertilized mouse oocytes. Four mice that carried the transgene at copy numbers ranging from about 2 to 30 were identified (FIG. 6b). Breeding studies demonstrated that all 4 founders transmitted the AHSP transgenes through the germ line to their offspring. Western blotting analysis using anti-V5 antibody demonstrated that AHSP-V5 was expressed at high levels in the spleen, a major site of red blood cell formation in mice (FIG. 6c). The expression levels correlated with transgene copy numbers.

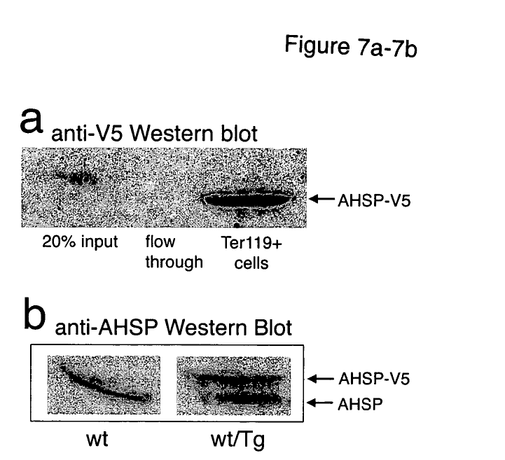

Using Magnetic Activated Cell Sorting (MACS), it was demonstrated that the AHSP-V5 protein is present mainly in erythrocytes, which are distinguished by expression of the cell surface marker Ter119 (FIG. 7a). An AHSP antibody detected both the endogenous and transgenic proteins (FIG. 7b). It was further determined that Line 1 (which has the highest copy number) expresses the transgene protein at roughly the same level as the endogenous, resulting in an overall doubling of the total amount of AHSP protein in the erythroid compartments.

Results/Discussion

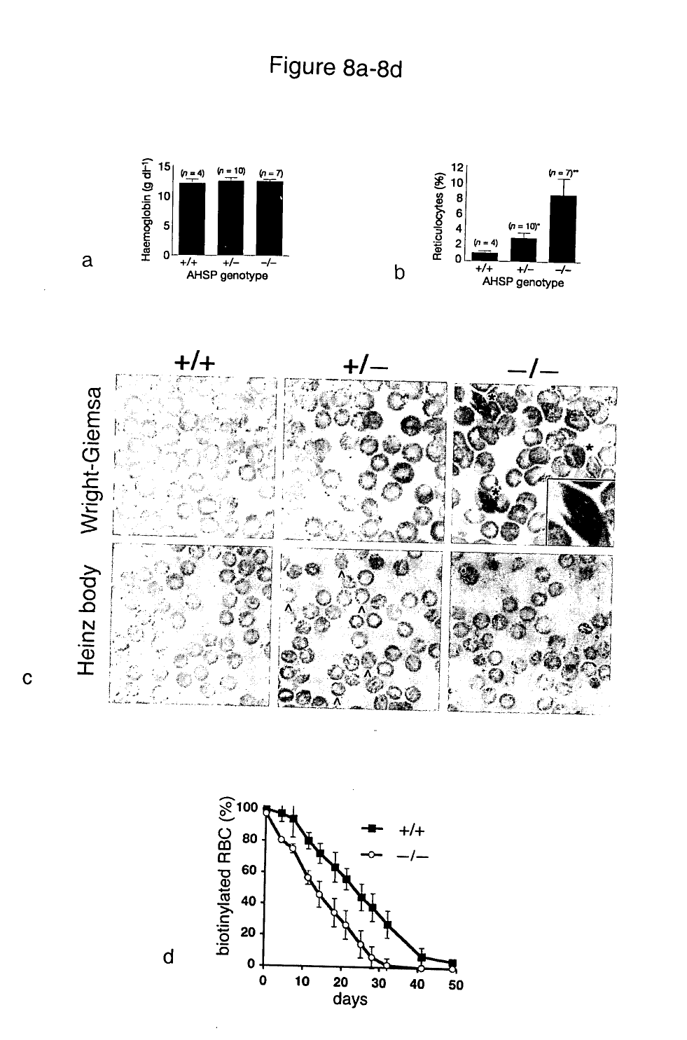

To investigate the requirement for AHSP in vivo, the entire protein-coding region of the gene in mice was deleted. AHSP^{-/-} mice were born at expected Mendelian ratios and displayed grossly normal growth and development (not shown). Although the mice exhibited normal blood hemoglobin concentrations and hematocrits (FIG. 8a, and not shown), their reticulocyte counts were elevated about threefold (FIG. 8b), indicating a shortened erythrocyte half-life. AHSP^{-/-} erythrocytes exhibited an abnormal spiculated morphology (FIG. 8c, top right panel) that has also been observed in erythroid cells of β-thalassaemic mice(Ciavatta et al., (1995) PNAS, Vol. 92, pages 9259-9263; Yang et al., (1995) PNAS, Vol. 92, pages 11608-11612.) Moreover, AHSP^{-/-} erythrocytes contained denatured hemoglobin inclusions (Heinz bodies) that stained with crystal violet (FIG. 8c, bottom panels). Thus, the loss of AHSP leads to defective hemoglobin metabolism in vivo. Considering the biochemical data, the erythrocyte abnormalities observed in AHSP^{-/-} mice probably result from a pathological accumulation of precipitated α-hemoglobin. In this regard, erythroid precursors contain a small pool of excess free α-hemoglobin (Shaeffer et al., (1967) Biochem. Biophys. Res. Comm., Vol. 28, pages 647-652; Tavill et al., (1967) Trans. Assoc. Am. Physic., Vol. 80, pages 305-313) whose stability and toxicity might be limited by direct interaction with AHSP. Thus, AHSP could interact transiently with free α-hemoglobin to maintain it in a quasi-native conformation before association with β-hemoglobin. The reticulocyte counts in AHSP^{±} mice were mildly elevated (FIG. 8b), indicating that AHSP haploinsufficiency might produce a subtle erythroid phenotype. It has recently been shown that AHSP (EDRF) mRNA is specifically downregulated in spleen, bone marrow and blood of animals with transmissible spongiform encephalopathies (TSEs, prion disease) (Miele et al., (2001) Nature Med. Vol. 7, pages 361-364.) Hematopoietic tissues are believed to participate in TSE pathogenesis. Accordingly, the role of AHSP in this process is also being investigated. TSEs are caused by refolding of the cellular protein PrPC into the disease-associated form, PrPSc, which forms toxic aggregates in the central nervous system (Prusiner et al., (1998) Cell., Vol. 93, pages 337-348.) In principle, this structural rearrangement could be influenced directly or indirectly by AHSP through its demonstrated ability to alter protein aggregation.

To further study AHSP expression, mice were created which are characterized by overexpression of AHSP. These mice provide superior in vivo models for the study of hemoglobin assembly, and for determination of therapeutic agents for disorders which are characterized by misassembly of hemoglobin, or the formation of plaques or inclusion bodies.

### Example III

### Oxidative Stress, Hemolytic Anemia, and Ineffective Erythropoesis Exhibited in AHSP KO Mice

As discussed previously, AHSP heterodimerizes with α Hb (Kd≅100 nM), but does not bind β Hb or Hb A. Moreover, α Hb bound to AHSP is highly resistant to oxidant-induced precipitation when compared to α Hb alone. Based on these findings, it was hypothesized that AHSP might protect erythroid cells from α Hb toxicity by maintaining α Hb in a stable state prior to its incorporation into Hb A. To test this, AHSP^{-/-} mice were generated by gene targeting, as described in Example II, above. Preliminary analysis of these animals revealed abnormal erythrocyte morphology with hemoglobin precipitates (Heinz bodies). These mutant mice were examined in greater detail to gain further insights into the molecular actions of AHSP in vivo.

As described below, it was found that loss of AHSP reduces the lifespan of circulating red blood cells and also causes increased apoptosis of erythroid precursors. These effects are mediated, at least in part, by increased production of ROS with consequent damage to Hb A and other cellular components. Moreover, ASHP blocks ROS production by α Hb directly. Together, these findings indicate that ASHP acts as protein-specific molecular chaperone that detoxifies free α Hb during normal erythropoiesis. In this capacity, altered AHSP levels could modulate the severity of pathological α Hb excess states, most importantly, β thalassemia.

Materials and Methods

Animals

Generation and genotyping of AHSP-deficient mice is described in Example II.

Hematologic Analysis

Mouse blood was sampled retro-orbitally, anticoagulated with EDTA and analyzed on a Bayer ADVIA 120 Hematology System. Unpaired student's t-tests were performed when analyzing the data.

For determination of erythrocyte half-life, N-hydroxysuccinimide (NHS)-biotin was injected intraperitoneally at 150 mg/kg on two consecutive days (days −2 and −1). 5 μl of blood was drawn from the tail vein, incubated with FITC-streptavidin (BD-PharMingen) and analyzed on a FACSCalibur (Hoffmann-Fezer, G., et al. (1993) Ann Hematol, 67, 81-87).

For phenylhydrazine treatment, drug was injected intraperitoneally at 63 mg/kg. Hematocrit and reticulocyte counts were analyzed for 9 days afterwards.

Methylcellulose colony assays to quantitate erythroid progenitors were performed as described (Keller, G., et al. (1993) Mol. Cell. Biol., 13, 472-486). 105 splenocytes or bone marrow cells were plated per 30 mM dish in triplicate. Cytokine combinations for specific progenitors were as follows: CFUe: Epo 2 U/ml; BFUe: Epo 2 U/ml and KL 50 ng/ml; myeloid: interleukin 3 (IL-3) 1 ng/ml, GM-CSF, 15 U/ml and M-CSF, 100 U/ml. Recombinant human Epo was purchased from Amgen. All other cytokines were murine and were purchased from R & D Systems.

Proteins

Recombinant AHSP and hemoglobins were purified as described (Gell, D., et al. (2002) J Biol Chem, 277, 40602-40609).

Analysis of Globin Precipitates

Heinz body stain was performed as previously described (Stamatoyannopoulos, G., et al.(1974) N Engl J Med, 290, 939-943). For globin chain analysis in membrane skeletons (Sorensen, S., et al. (1990) Blood, 75, 1333-1336), 40 μl of freshly-drawn blood was lysed and washed extensively in 0.05% PBS. Membrane lipids were removed by extraction with 56 mM Sodium Borate (pH 8.0). Precipitated globins were dissolved in 8M Urea, 10% Acetic Acid, 10% β-mercaptoethanol, 0.04% Pyronin, fractionated on Triton-Urea-Acetic acid (TAU) gels, and stained with Coomassie Briliant Blue. The fraction of sample loaded on the TAU gel was adjusted for the original hematocrit so that equal numbers of erythrocytes are represented in each lane.

Flow cytometry (FACS). Analysis to quantitate erythroid maturation stages in spleen and bone marrow was performed as previously described (Socolovsky, M., et al. (2001) Blood, 98, 3261-3273). Single cell suspensions were stained with PE-Ter119 and FITC-CD71 (BD PharMingen). 7-AAD (7-aminoactinomycin D, Sigma) was used to exclude dead cells. Studies were performed on a FACSCaliburTf System (Beckton-Dickinson). For detection of intracellular ROS, 10⁷ peripheral blood cells were pre-loaded with DCFH (2′,7′-dichlorofluorescein, Sigma) and then incubated with indicated concentrations of H₂O₂ and intracellular fluorescence intensity was detected by FACS (Zhu, H., et al. (1994). Arch Toxicol, 68, 582-587).

Detection of carbonyl-modified intracellular proteins. 20 μg of protein prepared from mouse erythrocytes was analyzed using the Oxyblot Kit (Intergen) as per the manufacturer's protocol. ROS production by α Hb. 40 μM α Hb ±40 μM AHSP was pre-incubated on ice for 30 minutes. TMPD (tetramethyl-p-phenylenediamine, Sigma) (Van der Ouderaa, F. J., et al. (1977) Biochim Biophys Acta, 487, 315-331) was then added to 400 μM final concentration and its oxidation measured by continuous light absorbance monitoring at 610 nm. The rate of oxidation was calculated based on the slope of the initial 10 minutes of the reaction. Heme loss was measured by the decrease of light absorbance in the Soret range (412 nm).

Hb A oxidation induced by its subunits. 5 μM α Hb was incubated on ice for 30 minutes in the presence or absence of 5 μM AHSP or SBTI (soybean trypsin inhibitor, Sigma) and then added to a mixture of 40 μM hemoglobin A and 1 mM GSH (Scott, M. D. and Eaton, J. W. (1995) Br J Haematol, 91, 811-819). The reaction was incubated at 37° C. for 30 minutes and the fraction of met (Fe III) Hb was calculated by linear regression to extinction coefficient spectra over the wavelength range 500-650 nm (Gow, A. J., et al. (1999) Proc Natl Acad Sci U S A, 96, 9027-9032).

Results

To further study the hematopoietic consequences of AHSP loss, the AHSP gene in mice was disrupted as described in Example II above. The targeting strategy replaced the entire protein-coding region with a PGK-Neo^{R} cassette flanked by loxP recombination sites. The hematopoietic abnormalities in AHSP^{-/-} animals reported below were identical when the PGK-Neo^{R} expression cassette was either present or removed by Cre-mediated recombination (not shown).

As described above, AHSP^{-/-} and AHSP^{±} mice were born at expected Mendelian ratios and displayed no gross abnormalities compared to wild type (AHSP^{+/+}) littermates. As expected, there was no AHSP RNA or protein detected in hematopoietic tissues of AHSP^{-/-} animals. Hematologic analysis of the gene-targeted mice performed using an automated analyzer (Bayer Advia 120) revealed several new findings not appreciated previously (Table II, below). No abnormalities in platelets or white blood cells were detected, but there were several obvious erythroid defects. AHSP^{-/-} animals exhibited a mild but significant anemia associated with small red blood cells (low mean cell volume, MCV) containing decreased Hb (low mean cell hemoglobin, MCH). There was significant variation in the size and hemoglobin content of the mutant erythrocytes as evidenced by increased red blood cell distribution width (RDW) and hemoglobin distribution width (HDW), respectively. The reticulocyte count was elevated in a subset of AHSP^{-/-} mice, indicating increased erythrocyte production to compensate for accelerated loss or destruction.

TABLE II
Altered erythrocyte indices of AHSP^{-/-} mice

| Index | AHSP^{+/+} (n = 8) | AHSP^{-/-} (n = 9) | p Value |
|---|---|---|---|
| Hb g/dL | 16.9 ± 0.6 | 14.5 ± 1.0 | <0.001 |
| Hct % | 55.8 ± 2.1 | 48.7 ± 3.2 | <0.001 |
| Hct % | 2.2 ± 1.0 | 4.4 ± 1.8 | =0.19 |
| MCV fL | 53.5 ± 1.6 | 45.6 ± 1.2 | <0.001 |
| MCH pg | 16.2 ± 0.5 | 13.6 ± 0.3 | <0.001 |
| RDW % | 13.1 ± 2.2 | 19.1 ± 2.0 | <0.001 |
| HDW g/dL | 1.5 ± 0.1 | 2.3 ± 0.1 | <0.001 |

Hb: hemoglobin;
Hct: hematocrit;
Retics: Reticulocytes;
MCV: Mean Corpuscular Volume;
MCH: Mean Corpuscular Hemoglobin;
RDW: Red Cell Distribution Width;
HDW: Hemoglobin Distribution Width.
Values shown are mean ± SD; n = number of mice analyzed.

The blood smears of AHSP^{-/-} mice showed numerous morphologic abnormalities including irregular size and shape, target cells and spiculated cells (FIG. 8C upper right panel). There were increased polychromatophilic cells representing newly synthesized erythrocytes; many of these contained eosinophilic inclusions. As mentioned previously, inclusion bodies were also apparent in AHSP^{-/-} erythrocytes after staining with crystal violet, which detects denatured globin chains (Heinz bodies, FIG. 8C, lower panels). Of note, AHSP^{±} erythrocytes also contained occasional Heinz bodies, suggesting haploinsufficiency effects (see below). Unstable denatured Hbs can accelerate erythrocyte destruction by causing intravascular hemolysis and/or sequestration by the reticuloendothelial system. In support of this, the presence of large inclusions only in polychromatophilic cells suggests that these are rapidly cleared from the circulation. This preferential loss of nascent erythroid cells could result in a reticulocyte count that is lower than expected for the degree of erythrocyte destruction.

To determine the effects of AHSP loss on erythrocyte lifespan more directly, animals were injected with N-hydroxysuccinimide (NHS)-biotin, which labeled nearly all circulating red blood cells with biotin (FIG. 8D). The survival of tagged erythrocytes was then followed over a 50-day period by removing small samples (˜5 μl) of blood from the tail vein, staining with FITC-PE, and quantitating the fraction of labeled cells using flow cytometry. Erythrocytes from normal littermates exhibited a half-life of about 22 days, in accord with previous studies (Hoffmann-Fezer, G., et al. (1993) Ann Hematol, 67, 81-87). In contrast, the half-life of AHSP^{-/-} erythrocytes was significantly shortened to about 12 days. Hence, loss of AHSP causes significant premature destruction of circulating red blood cells. The observed differences in clearance of wild-type and mutant erythrocytes may be underestimated, since circulating erythroid cells of mutant animals are relatively younger due to increased erythropoiesis.

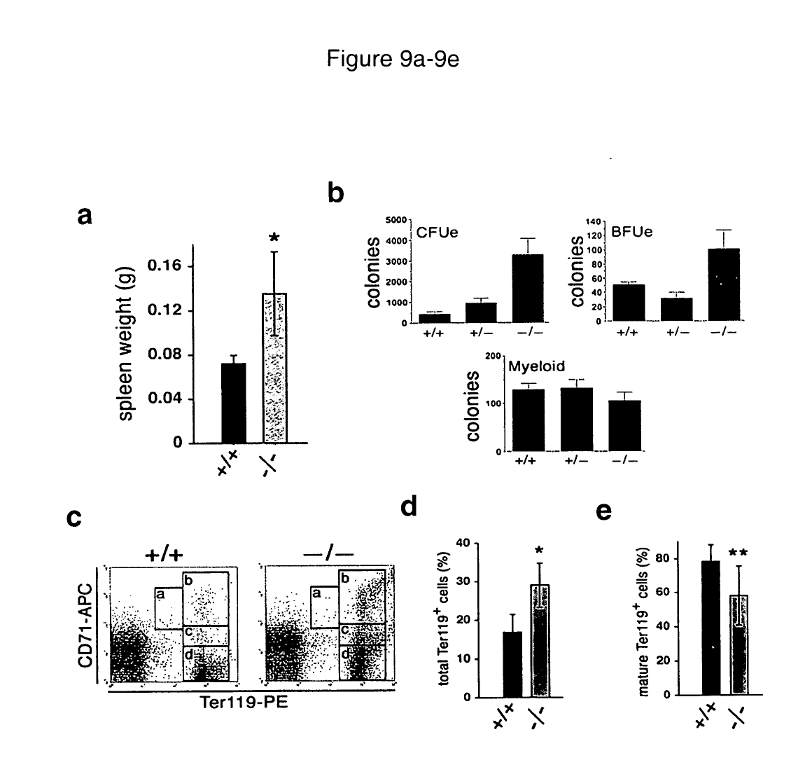

In most disorders of erythrocyte destruction, there is a compensatory increase in production of erythroid precursors in hematopoietic tissues. The spleens, a major site of erythropoiesis in mice, were significantly enlarged in AHSP^{-/-} mice (FIG. 9A). To quantitate erythroid precursors, spleens were disaggregated into single cell suspensions and seeded into semisolid medium. This assay assesses the developmental potential of individual cells and identifies two types of committed erythroid precursors: Burst forming unit erythroid (BFUe) cells represent early-stage precursors which give rise to large hemoglobinized colonies in the presence of erythropoietin (Epo) and stem cell factor (SCF). Colony forming unit erythroid (CFUe) precursors represent a later stage of development and give rise to smaller colonies in the presence of Epo alone. As shown in FIG. 9B, the proportions of both BFUe and CFUe precursors were elevated in AHSP^{-/-} mice. In contrast, myeloid progenitors, which give rise to granulocyte and macrophage-containing colonies in appropriate cytokines (see Methods), were unchanged. Therefore, splenic enlargement observed in the mutant animals is due to expansion of the erythroid compartment. Similar findings were obtained upon analysis of bone marrow (not shown). These results reflect increased erythropoietic drive to compensate for accelerated destruction of mature AHSP^{-/-} erythrocytes.

In β thalassemia, accumulation of excess free α Hb not only damages mature erythrocytes, but also induces apoptosis of erythroid precursors in hematopoietic tissues in a process termed ineffective erythropoiesis (Rachmilewitz, E. and Schrier, S. L. (2001) Pathophysiology of b thalassemia. In Disorders of Hemoglobin. Cambridge University Press, Cambridge, pp. 233-251). To investigate whether loss of AHSP causes ineffective erythropoiesis, splenocytes were examined for coexpression of the erythroid marker Ter 119 and the transferrin receptor CD71, which subdivides erythroid precursors according to their maturation stage (FIG. 9C) (Socolovsky, M., et al. (2001) Blood, 98, 3261-3273). The proportion of Ter 119+cells was elevated in AHSP^{-/-} spleens (FIG. 9D), consistent with erythroid hyperplasia described above. However, the AHSP^{-/-} animals exhibited an elevated proportion of immature erythroid precursors (Ter119 high, CD71 high) compared to mature ones (Ter119 high, CD71 low), suggesting that the transition from immature to mature precursor was partially blocked (FIGS. 9C and 9E). This effect could reflect ineffective erythropoiesis. To determine this, erythroid precursor apoptosis was quantified by direct immunohistochemical examination (FIG. 9F). Consistent with the flow cytometry data, AHSP^{-/-} spleens contained increased Ter119-staining cells. Moreover, apoptosis of erythroid precursors was significantly elevated in the mutant animals, as evidenced by increased proportion of erythroid (Ter119+) cells exhibiting endonucleolytic DNA cleavage (TUNEL+). Hence, loss of AHSP not only damages mature erythrocytes, but it is also toxic to erythroid precursors, similar to the effects of excess α Hb in β thalassemia and consistent with a role for AHSP stabilizing free α Hb.

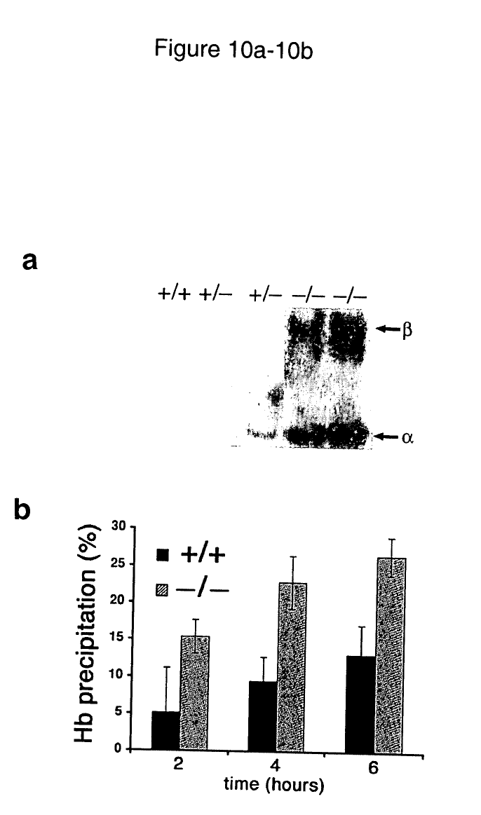

Loss of AHSP results in both α and β globin precipitates. Next, the globin precipitates in AHSP^{-/-} mice were characterized. In disorders associated with unstable hemoglobins, including thalassemias, denatured globins precipitate onto the cell membrane and damage associated lipids and proteins (Joshi, W., et al. (1983) J Lab Clin Med, 102, 46-52; Weatherall, D. J., et al. (1969) Br J Haematol, 16, 251-267; Shinar, E., et al. (1989) J Clin Invest, 83, 404-410; Shinar, E., et al. (1987) Blood, 70, 158-164; Rouyer-Fessard, P., et al. (1989) J Biol Chem, 264, 19092-19098; Schrier, S. L., et al. (1989) Blood, 74, 2194-2202; Advani, R., et al. (1992) Blood, 79, 1058-1063). Plasma membranes were isolated from mutant erythrocytes and wild-type littermates, extracted with detergent to remove lipids and analyzed the resultant membrane skeletons for associated globins by triton-acid urea gel electrophoresis (FIG. 10A). While erythrocyte membrane-associated globin chains were never observed in wild-type animals, about half of the AHSP^{±} mice examined showed low-level a globin precipitate, consistent with the presence of occasional Heinz bodies noted in FIG. 8C, above. Surprisingly, AHSP^{-/-} erythrocytes contained both a and f globin precipitates at roughly equimolar ratios, despite the previous findings that AHSP specifically binds and stabilizes α Hb. One mechanism to reconcile this apparent paradox is that excess free α Hb can produce ROS that oxidize and destabilize Hb A (Scott, M. D. and Eaton, J. W. (1995) Br J Haematol, 91, 811-819; Scott, M. D., et al. (1990) J Biol Chem, 265, 17953-17959; Scott, M. D., et al. (1993) J Clin Invest, 91, 1706-1712), eventually causing it to precipitate. In this case, unstable Hb A should be present in AHSP^{-/-} erythrocytes. To test this, soluble Hb A was prepared from fresh hemolysates and it's stability was examined after addition of 17% isopropanol, which causes preferential precipitation of unstable hemoglobins (Kim, H. C. and Schwartz, E. (1995) Unstable Hemoglobins. In Williams Hematology. McGraw Hill, New York, pp. L33-L34). As illustrated in FIG. 10B, soluble Hb A from AHSP^{-/-} erythrocytes precipitated more readily in isopropanol, suggesting prior damage. Taken together with the prior biochemical data, these findings indicate that loss of AHSP destabilizes α Hb, which then leads to oxidation and eventual precipitation of Hb A. While occasional Heinz bodies and low-level a globin precipitates were observed in AHSP^{±} animals, erythroid indices and circulating lifespans were normal (not shown). This suggests that AHSP haploinsufficiency destabilizes α Hb, but not enough to exceed a threshold required for Hb A damage and significant hemolysis.

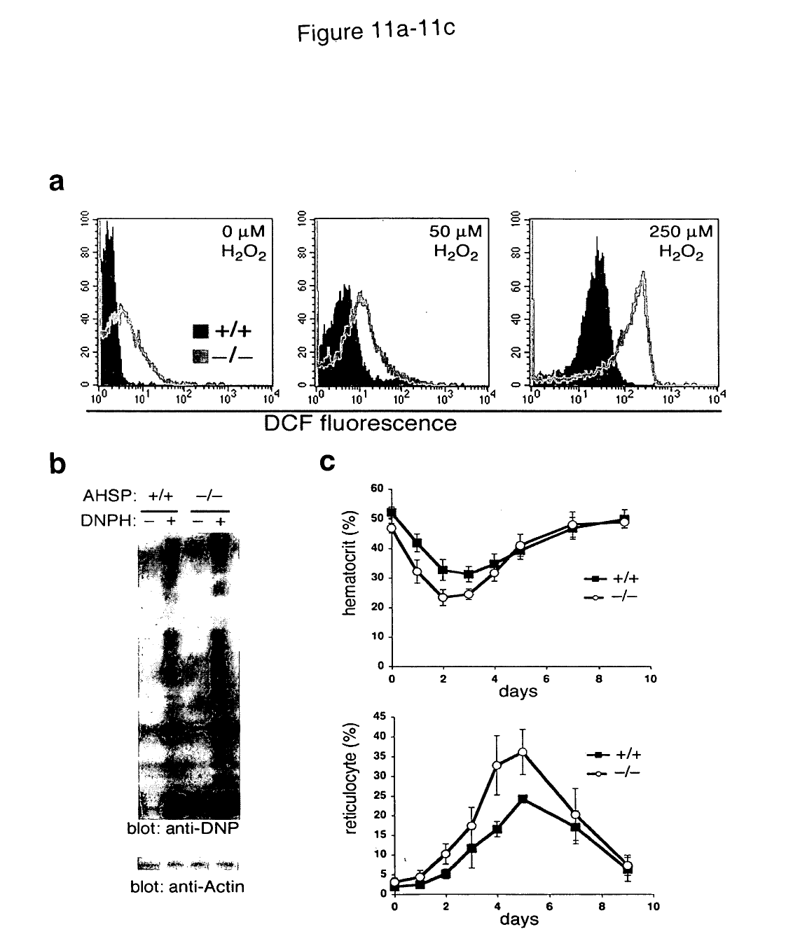

It is possible that many of the abnormalities observed in AHSP^{-/-} erythroid cells are caused by ROS generated from destabilized α Hb, a potent oxidant. This was investigated by incubating erythroid cells with 2′, 7′ dichloro dihydrofluroscein (DCFH), an ROS indicator that is converted to a fluorescent product upon oxidation. As predicted, mutant erythrocytes contained increased ROS both at baseline and after challenge with hydrogen peroxide, a physiologic ROS precursor (FIG. 11A). One consequence of oxidative damage to proteins is introduction of carbonyl groups onto amino acid side chains. This can be examined by derivatizing protein-associated carbonyl moieties with 2,4-dinitrophenylhydrazine (DNPH), followed by Western blot analysis using an antibody that detects the attached DNP group (Nakamura, A. and Goto, S. (1996) J Biochem (Tokyo), 119, 768-774). Using this approach, it was found that AHSP^{-/-} erythrocytes exhibited distinct oxidative damage, as reflected by elevated levels of carbonyl groups on numerous proteins (FIG. 11B).

Erythrocytes with intrinsically elevated oxidative stress exhibit baseline damage and depletion of endogenous antioxidant defenses, and therefore, are expected to be more sensitive to injury from external oxidants. AHSP^{-/-} mice were challenged with phenylhydrazine, an oxidant causing hemolytic anemia to test this hypothesis. Compared to wild-type littermates, mutant animals exhibited a more dramatic fall in hematocrit after treatment with the drug (FIG. 11C, upper panel). Hence, loss of AHSP renders erythrocytes more susceptible to oxidative damage. Of note, the mutant animals responded appropriately to acute anemia by inducing a rapid reticulocyte response (FIG. 11C, lower panel). Hence, the partial block to erythroid development in AHSP^{-/-} mice described in FIG. 10, above is not severe enough to impair physiologic responses to moderate acute anemia.

As described above, biochemical studies demonstrated that AHSP binds and stabilizes α Hb specifically. Free α Hb produces ROS by several known mechanisms and the resultant oxidative damage to erythroid cells is believed to be a major determinant of pathophysiology in α Hb-excess states, most importantly, β thalassemia (Schrier, S. L. (2002) Curr Opin Hematol, 9, 123-126). AHSP could limit damage from α Hb by inhibiting its ability to produce ROS.

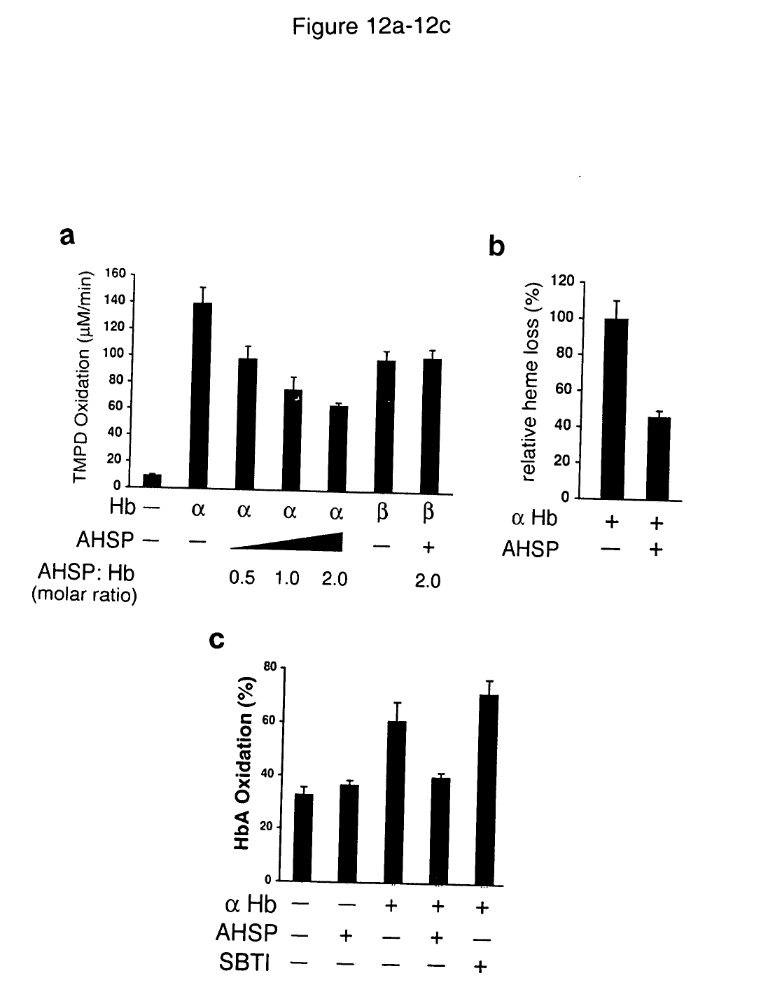

Heme-containing proteins, including purified a or β Hb subunits, catalyze the production of ROS from hydrogen peroxide (H₂O₂), in a process that can be quantified using N, N, N′, N′-tetramethyl-p-phenylene diamine (TMPD), a ROS-sensitive dye that fluoresces upon oxidation (Van der Ouderaa, F. J., et al. (1977) Biochim Biophys Acta, 487, 315-331) (FIG. 12A). To assess the effects of AHSP on this process, heme proteins were preincubated with recombinant purified AHSP for 30 minutes prior to addition of H₂O₂ and TMPD. Remarkably, AHSP, which binds α Hb at 1:1 stoichiometry (Gell, D., et al. (2002) J Biol Chem, 277, 40602-40609), inhibited ROS production by α Hb in a dose-dependent fashion. These effects were specific, as AHSP did not inhibit formation of ROS from β Hb or myoglobin (FIG. 12A and not shown). Hence, one mechanism through which AHSP detoxifies α Hb is to suppress its pro-oxidant activities.

Oxidation of the heme iron of α Hb to the ferric form allows it to participate in further redox reactions that can result in protein destabilization and release of the heme moiety. This is reflected by reduced absorbance in the Soret peak at 412 nm (not shown). Preincubation of α Hb with AHSP inhibited H₂O₂-induced loss of heme, indicating that protein destabilization associated with the oxidative process was reduced (FIG. 12B). This result is in accord with AHSP-mediated suppression of ROS production by α Hb (FIG. 12A, above) and with the previous findings that AHSP protects α Hb from oxidant-induced precipitation (above).

It is possible that relatively low levels of unstable a Hb in AHSP^{-/-} erythrocytes propagate ROS production with consequent oxidation and precipitation of other Hb A and other cellular components. This would explain the presence of β globin precipitates and unstable Hb A in mutant erythrocytes (FIG. 10). To test this, the ability of free α Hb to promote Hb A oxidation in solution was examined (FIG. 12C). Conversion of Hb A to the oxidized met (Fe III) form is accompanied by characteristic absorbance changes in the visible spectrum that was monitored via deconvolution to known extinction coefficient spectra. After 30 minutes incubation at 37° C., ˜35% of oxy Hb A was converted to the met form; this was not affected by addition of AHSP (FIG. 12C). Addition of substoichiometric free α Hb (0.125 molar ratio) roughly doubled the amount of Hb A oxidation. Hence, free α Hb can promote Hb A oxidation, presumably through production of ROS. These findings are consistent with those of Scott et al who showed that introducing free α Hb into normal erythrocytes promotes Hb A oxidation (Scott, M. D., et al. (1990) J Biol Chem, 265, 17953-17959; Scott, M. D., et al. (1993) J Clin Invest, 91, 1706-1712). Remarkably, preincubation with AHSP strongly inhibited the ability of free α Hb to induce oxidation of Hb A. In contrast, soybean trypsin inhibitor (STBI), a control protein of similar size to AHSP, had no effect on α Hb-mediated Hb A oxidation. Therefore, AHSP specifically and directly inhibits the oxidation of Hb A by a Hb. These findings are consistent with the ability of AHSP to inhibit ROS production by α Hb and also help to explain the pathophysiology of unstable Hb A in AHSP^{-/-} erythrocytes (FIGS. 8 and 10).

Discussion

Erythrocyte precursors synthesize massive amounts of Hb A tetramer, yet manage to avoid damage from cytotoxic free heme and globin subunit precursors. The protective mechanisms associated with Hb A synthesis are beginning to be understood. For example, evidence for molecular crosstalk to balance Hb A precursor production is provided by heme-regulated kinase (HRI), a sensor that inhibits translation of globin chains when heme availability is limited (Han, A. P., et al. (2001) Embo J, 20, 6909-6918). In addition, proteolytic pathways to degrade excess free α Hb are present in erythroid cells (Shaeffer, J. R. (1983) Journal of Biological Chemistry, 258, 13172-13177; Shaeffer, J. R. and Cohen, R. E. (1998) Ann N Y Acad Sci, 850, 394-397; Shaeffer, J. R. and Kania, M. A. (1995) Biochemistry, 34, 4015-4021). It is also to be expected that molecular chaperones exist to transiently stabilize Hb A synthetic intermediates prior to their assembly. α Hb exists as a monomer, rendering it particularly unstable compared to D Hb and Hb A, which exist mainly as tetramers under physiologic conditions. For this reason, a chaperone for α Hb would be particularly advantageous; evidence provided herein indicates that AHSP serves this function.

AHSP is abundant in late stage erythroid precursors where its expression kinetics parallel that of a globin (dos Santos, C. O., et al. (2004) Experimental Hematology, 32, 157-162). AHSP specifically binds free α Hb in a fashion that is not expected to interfere with Hb A assembly or function (Gell, D., et al. (2002) J Biol Chem, 277, 40602-40609). Moreover, AHSP-bound α Hb is resistant to denaturation and can still be incorporated into Hb A when β Hb is not limiting (Kihm, A. J., et al. (2002) Nature, 417, 758-763). These observations are consistent with a model in which AHSP serves as a docking molecule to temporarily bind α Hb, stabilize its structure and render it biochemically inert prior to Hb A assembly.

The toxicities of α Hb are most thoroughly studied in the context of β thalassemia where unpaired α Hb precipitates reduce the survival of circulating erythrocytes and also cause ineffective erythropoiesis associated with impaired viability and apoptosis of erythroid precursors. Similarly, AHSP^{-/-} mice exhibit shortened erythrocyte half-life with globin precipitates and ineffective erythropoiesis. Unpaired α Hb is a potent oxidant and β thalassemic erythrocytes exhibit increased ROS, depleted endogenous antioxidants and pathological oxidation of cellular lipids and proteins (reviewed in Shinar, E. and Rachmilewitz, E. A. (1990) Semin Hematol, 27, 70-82). AHSP^{-/-} erythrocytes also contain elevated ROS and signs of oxidative damage. Hence, β thalassemia and AHSP deficiency share many pathological features. Based on these findings and the specificity of AHSP for α Hb, the erythroid defects in AHSP^{-/-} mice likely stem from destabilized α Hb.

Previous studies (Brunori, M., et al. (1975) Eur. J. Biochem., 53, 99-104; Scott, M. D. and Eaton, J. W. (1995) Br J Haematol, 91, 811-819; Scott, M. D., et al. (1990) J Biol Chem, 265, 17953-17959; Scott, M. D., et al. (1993) J Clin Invest, 91, 1706-1712) and the current data (FIG. 11) demonstrate that free α Hb generates ROS that can lead to oxidation of Hb A. Remarkably, AHSP inhibits both of these processes directly in solution. It is likely that loss of AHSP in vivo leads to increased production of ROS from α Hb, which in turn, damage Hb A and other cellular constituents. This mechanism probably explains why both α and β globin precipitates are present in the erythrocyte membrane of AHSP^{-/-} mice. In contrast, erythrocytes from mice and humans with β thalassemia contain mainly a globin precipitates, despite high levels of ROS that might be expected to damage Hb A. This difference could be because reduced P chain synthesis in β thalassemia favors α Hb precipitation. In addition, damaged Hb A may be less likely to precipitate at abnormally low concentrations present in β thalassemia.

AHSP could interfere with ROS production by α Hb through one or more interrelated mechanisms (reviewed in Shinar, E. and Rachmilewitz, E. A. (1990) Semin Hematol, 27, 70-82; Bunn, H. F. and Forget, B. G. (1986) Unstable Hemoglobin Variants-Congenital Heinz Body Hemolytic Anemia. In Hemoglobin: Molecular, Genetic, and Clinical Aspects. W.B. Saunders, Philadelphia, pp. 565-594). Oxy- α Hb is particularly predisposed to generate ROS in a process termed autoxidation, which occurs upon heterolytic cleavage of the iron-oxygen bond to yield superoxide anion (O₂⁻) and met (Fe III) Hb. O₂⁻ is readily converted to hydrogen peroxide (H₂O₂) and water, a process catalyzed in erythrocytes by superoxide dismutase. In the presence of redox-active metals, such as iron, H₂O₂ is converted to the potent oxidant, hydroxyl radical. This reaction can occur in the context of free α Hb in the heme peroxidase reaction (Sadrzadeh, S. M., et al. (1984) J Biol Chem, 259, 14354-14356) or also be catalyzed by free iron or heme, as in the Fenton reaction. In addition, oxidized α Hb is intrinsically unstable and undergoes a series of rearrangements in which amino acid sidegroups surrounding the heme-binding pocket become covalently attached to heme-bound iron. These structures, termed hemichromes and their ultimate degradation products, free heme, porphyrins and iron are all potent catalysts of redox chemistry, and hence, ROS production. AHSP could block ROS production from α Hb by reducing its inherent ability to participate in redox reactions and/or by inhibiting its subsequent conversion to unstable hemichromes.

Of note, some AHSP^{±} (heterozygous) erythrocytes contained low level a globin precipitates and Heinz bodies. However, AHSP^{±} animals were hematologically normal and did not contain elevated ROS or precipitated β globin chains in their erythrocytes (not shown). These findings indicate that AHSP haploinsufficiency causes some destabilization of α Hb, but does not appear to exceed a threshold level required for clinically significant erythroid pathology. However, it is possible that AHSP haploinsufficiency may be clinically significant under conditions of oxidative stress or pathologic α Hb excess, such as in β thalassemia (see below) or triplicated a globin genes.

The findings that AHSP specifically mitigates α Hb-associated toxicities, both biochemically and in vivo, have several potential implications for human disease. For example, defects in AHSP could be responsible for unexplained cases of Heinz body hemolytic anemia in humans. Loss of AHSP bears some similarities to known erythrocyte disorders including mild β thalassemia and glucose-6-phosphate dehydrogenase deficiency, which are believed to be protective against malaria (Weatherall, D. J. and Clegg, J. B. (2002) Genes Immun, 3, 331-337; Cooke, G. S. and Hill, A. V. (2001) Nat Rev Genet, 2, 967-977). If similar advantageous effects are conferred by AHSP deficiency, then mutant alleles could be prevalent through genetic selection in malarious regions. This possibility can now be tested by examining malaria susceptibility in AHSP^{-/-} mice and also by analyzing genomic AHSP DNA in selected human populations. In addition, numerous missense mutations in the a globin gene are known to destabilize the resultant protein and cause hemolytic anemia (Beutler, E. (2001) Hemoglobinopathies Associated With Unstable Hemoglobin. In Williams Hematology. McGraw Hill, New York). It is possible that some of these pathologic human mutations destabilize α Hb by inhibiting its ability to interact with AHSP.

Finally, variations in AHSP levels could modulate the severity of erythrocyte instability or ineffective erythropoiesis in β thalassemia where the extent of excess free α Hb contributes significantly to disease pathophysiology. In support of this, preliminary findings in mice indicate that loss of AHSP worsens the β thalassemia phenotype (Kong, Y., et al. (2003) Blood, 102, 46a).

### Example IV

### Role of AHSP in Hemoglobin Production and/or Metabolism

The previous examples provide evidence that AHSP functions at several stages during hemoglobin production or metabolism. For example, it may act as a molecular chaperone to facilitate alpha chain synthesis, a model supported by the observation that AHSP also stabilizes apo-alpha globin. In addition, AHSP may stabilize the small pool of excess free alpha hemoglobin previously demonstrated to exist during normal erythropoiesis. Both of these potential functions predict specific molecular interactions between AHSP and alpha or beta thalassemia. To analyze the effects of globin gene dosage on the AHSP phenotype described herein (and vice versa), AHSP gene-deleted mice were interbred with animals containing defined alpha or beta globin deficiencies.

Interaction Between AHSP and Beta Thalassemia

To investigate AHSP and the severity of beta thalassemia, a model in which both beta major and minor globins are deleted from a single chromosome was used. Heterozygous animals have thalassemia intermedia while homozygotes are embryonic lethal. The effects of AHSP loss on the heterozygous animals was studied. To generate test animals, AHSP null mice were intercrossed with transgenic mice having thalassemia intermedia. The F1 double heterozygotes were then crossed to obtain AHSP-null beta thalassemic animals.

Of the beta thalassemic offspring, all AHSP wt mice survive to adulthood. In contrast, the betα-thalassemic/AHSP-null combination results in partially-penentrant embryonic lethality, in which two-thirds of animals die in utero and the remaining one third survive to adulthood.

The surviving AHSP-null thalassemic animals are significantly more anemic, showing a 20% decrease in hematocrit levels. The size of the erythrocytes from these compound mutants is also more variable, as evidenced by increased red blood cell distribution width, (RDW).

In conclusion, loss of AHSP results in partially-penetrate embryonic lethality in beta thalassemic mice with the surviving animals being more anemic. The ability of AHSP to stabilize excess free alpha globin present in beta thalassemia could account for these findings. In this model, free alpha globin is more rapidly destabilized in the absence of AHSP. This accelerates cytotoxic alpha globin precipitation, and the generation of damaging reactive oxygen species or ROS. Hence, AHSP loss exacerbates beta thalassemia by destabilizing free alpha globin.

Interaction Between AHSP and Alpha Thalassemia

Next, the phenotype of transgenic mice resulting from the cross of AHSP^{-/-} mice, and mice lacking an alpha globin gene was examined.

The alpha thalassemic mice have 3 of 4 alpha genes present, and are called “silent carriers’. The animals are phenotypically normal except for mild microcytosis. The AHSP-null animals were interbred with alpha thalassemic mice. Then F1 double heterozygotes were intercrossed to examine whether alpha globin gene dosage modifies the AHSP null phenotype or vice versa.

Based on previous finding that loss of AHSP exacerbates beta thalassemia, it was predicted that slightly decreased alpha globin chain ratio should alleviate AHSP-null phenotype. Surprisingly, it was found that the combination of the two mutations resulted in a dramatically impaired hemoglobin synthesis and stabilization. Double mutant mice, appeared to be more anemic, exhibited lower hematocrits (by 20%), higher reticulocyte counts, decreased Mean Corpuscular Volume, and increased RDW. Inspection of the blood smears showed that double mutant erythrocytes were more hypochromic with greater variability in size and shape, compared to AHSP null or alpha thalassemia silent carrier erythrocytes. There were also increased numbers of target cells (red blood cells with pooled hemoglobin in the center, which look like “targets”), as well as polychromotophilic cells containing inclusions, suggesting increased globin precipitation. Therefore, reduced alpha globin gene dosage worsens AHSP deficiency and furthermore, AHSP deficiency worsens alpha thalassemia in mice.

This finding is consistent with the observation that loss of AHSP impairs alpha globin synthesis. It is believed that AHSP can bind and stabilize nascent alpha globin, either on the ribosome or immediately after its release. Loss of AHSP results in alpha globin instability and precipitation. Consequently, there is increased beta globin excess which potentially allows more free beta globin to accumulate in alpha thalassemia. Therefore, loss of AHSP exacerbates alpha thalassemia by worsening chain imbalance.

In summary, loss of AHSP worsens beta thalassemia, which further provides evidence of its role in stabilizing excess free alpha globin. Additionally, loss of AHSP also worsens alpha thalassemia, which provides evidence that AHSP also functions to enhance alpha globin synthesis. Clearly, AHSP has multiple functions during hemoglobin synthesis and the relative importance of these functions depends on globin chain ratios. Accordingly the mice described above provide ideal in-vivo model systems for assessing the efficacy of agents for the treatment of alpha and betα-thalassemias, as well as other anemias.

### Example V

### Use of AHSP Knockout Mice for the Generation of AHSP Specific Antibodies

Alpha hemoglobin stabilizing protein is an abundant protein that has been highly conserved throughout evolution. The extensive similarity of AHSP proteins among species presents certain problems for the generation of alpha hemoglobin stabilizing protein-specific monoclonal antibodies.

Antibodies are generated in response to exposure to foreign antigens. The foreign antigens must be recognized as “non-self” before an immune response will be mounted. The AHSP knock out mice of the invention can be used to advantage for the production of AHSP antibodies as these animals do not express native alpha hemoglobin stabilizing protein. Production of antibodies in AHSP mice will produce a more diverse array of epitope specificities than generation of antibodies in mice which express native AHSP. Antibodies so generated will provide a useful research tool for intracellular localizations, epitope mapping, and immunoprecipitation studies for characterizing those proteins that form intracellular associations with alpha hemoglobin stabilizing protein. Utilization of knock out mice for this purpose ensures that the immunizing protein antigen will be recognized as non-self and therefore invoke a powerful immune response. Antibodies to AHSP orthologs from different species may be generated. For example, some AHSP orthologs include, but are not limited to Mus musculus, Accession No. AF485327, Bos tarus, Accession No. AF485326, Homo Sapien, Accession No. AF485325, Rattus Norvegicus, Accession No. XM_{—}215059, and Sus scrofa, Accession No. AW480086.

Additional potential applications for the antibodies of the invention include assays to determine whether a particular epitope on the alpha hemoglobin stabilizing protein has been modified. Such antibodies may be used to advantage to assess post-translational modifications or modifications associated with a particular disease state, such as particular anemias, and transmissible spongiform encephalopathies. The monoclonal antibodies of the invention may also be used to quantify various species of alpha hemoglobin stabilizing proteins, for example in ELISA reactions. It is likely that AHSP levels in plasma deviate from normal with particular disease states. For example, decreased levels of AHSP are associated with various anemias, and with spongiform encephalopathies. Thus, the ability to easily and accurately quantify AHSP levels would be clinically useful. Finally, the antibodies of the invention may also be utilized to inhibit or alter alpha hemoglobin stabilizing protein action.

Polyclonal antibodies can be raised by administration of alpha hemoglobin stabilizing protein to the knockout mice of the invention, using known immunization procedures. Usually a buffered solution of the antigen accompanied by Freund's adjuvant is injected subcutaneously at multiple sites. A number of such administrations at intervals of days or weeks is usually necessary. A number of knockout mice, for example from 3 to 20, are so treated with the expectation that only a small proportion will produce good antibodies. The antibodies are recovered from the knockout mice after some weeks or months.

The use of monoclonal antibodies is particularly preferred because they can be produced in large quantities and the product is homogeneous. The preparation of hybridoma cell lines for monoclonal antibody production derived by fusing an “immortal” cell line and lymphocytes sensitized against the immunogenic preparation can be done by techniques which are well known to those who are skilled in the art. See, for example, Doullard, J. Y. and Hoffman, T., “Basic Facts About Hybridomas” in Compendium of Immunology, vol. II, L. Schwartz (ed.) (1981); Kohler, G. and Milstein, C., Nature, 256:495-497 (1975); Koprowski, et al., European Journal of Immunology, 6:511-519; Koprowski et al., U.S. Pat. No. 4,172,124; Koprowski et al., U.S. Pat. No. 4,196,265; and Wands, U.S. Pat. No. 4,271,145; the teachings of which are herein incorporated by reference.

For the purpose of the present invention, the AHSP knockout mice may be injected with approximately 0.1 mg to about 20 mg of purified AHSP or fragments thereof. Usually the injecting material is emulsified in Freund's complete adjuvant. Boosting injections may also be required. The detection of antibody production can be carried out by testing the antisera with appropriately labeled antigen, as required by radioimmunoprecipitation, or with capture complex, as required by a variety of solid phase immunoassays including competitive ELISA. Lymphocytes can be obtained by removing the spleen or lymph nodes of sensitized animals in a sterile fashion and carrying out cell fusion. Alternatively, lymphocytes can be stimulated or immunized in vitro, as described, for example, in C. Reading, J. Immunol. Meth., 53:261-291, (1982).

A number of cell lines suitable for fusion have been developed, and the choice of any particular line for hybridization protocols is directed by any one of a number of criteria such as speed, uniformity of growth characteristics, absence of immunoglobulin production and secretion by the nonfused cell line, deficiency of metabolism for a component of the growth medium, and potential for good fusion frequency.

Intraspecies hybrids, particularly between like strains, work better than interspecies fusions. Several cell lines are available, including mutants selected for the loss of ability to secrete myeloma immunoglobulin. Included among these are the following mouse myeloma lines: MPC sub 11-X45-6TG, P3-NS1-1-Ag4-1. P3-X63-Ag8, or mutants thereof such as X63-Ag8.653, SP2-O-Ag14 (all BALB/c derived), Y3-Ag1.2.3 (rat) and U266 (human).

Cell fusion can be induced either by virus, such as Epstein-Barr or Sendai virus, or by polyethylene glycol. Polyethylene glycol (PEG) is the most efficacious agent for the fusion of mammalian somatic cells. PEG itself may be toxic for cells, and various concentrations should be tested for effects on viability before attempting fusion. The molecular weight range of PEG may be varied from 1000 to 6000 da. The ratio between lymphocytes and malignant cells is optimized to reduce cell fusion among spleen cells and a range of from about 1:1 to about 1:10 (malignant cells:lymphocytes) gives good results.

The successfully fused cells can be separated from the myeloma line by any technique known in the art. The most common and preferred method is to choose a malignant line which is Hypoxanthine-Guanine Phosphoribosyltransferase (HGPRT) deficient. These cells will not grow in an aminopterin-containing medium. HAT medium is generally composed of hypoxanthine 1×10⁻⁴ M, aminopterin 4×10⁻⁷ M, and thymidine 1.6×10⁻⁵ M. The use of such a medium facilitates the rapid selection of hybrid cells. The fusion mixture can be grown in the HAT-containing culture medium immediately after the fusion. Cell culture usually entails maintenance in HAT medium for one week and then feeding with either regular culture medium or hypoxanthine, thymidine-containing medium.

The growing colonies are then tested for the presence of antibodies that recognize alpha hemoglobin stabilizing protein. Detection of hybridoma antibodies can be performed using an assay where the capture complex is bound to a solid support and allowed to react with hybridoma supernatants containing putative antibodies. The presence of antibodies may be detected by direct ELISA techniques using a variety of indicators. Most of the common methods are sufficiently sensitive for use in the range of antibody concentrations secreted during hybrid growth.

The aforementioned techniques may be used to generate antibodies to alpha hemoglobin stabilizing protein (AHSP) and homologs (including orthologs), or fragments thereof. Certain AHSP orthologs include, but are not limited to Mus musculus, Accession No. AF485327, Bos tarus, Accession No. AF485326, Homo Sapien, Accession No. AF485325, Rattus Norvegicus, Accession No. XM_{—}215059, and Sus scrofa, Accession No. AW480086.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. A transgenic mouse harboring a homozygous null mutation in its endogenous Alpha Hemoglobin Stabilizing Protein (AHSP) gene wherein said mouse does not express a functional mouse Alpha Hemoglobin Stabilizing Protein (AHSP) protein and erythrocytes obtained from said mouse exhibit one or more characteristics selected from the group consisting of abnormal spiculated morphology, reduced lifespan, increased production of reactive oxygen species (ROS), and precipitated hemoglobin.

2. The transgenic mouse of claim 1, wherein said mouse is fertile and transmits said null mutation to its offspring.

3. The transgenic mouse of claim 1, wherein said null mutation has been introduced into an ancestor of said mouse at an embryonic stage following microinjection of embryonic stem cells into a mouse blastocyst.

4. The transgenic mouse of claim 1, wherein said null mutation has been introduced into an ancestor of said mouse at an embryonic stage following co-incubation of embryonic stem cells with a fertilized egg or morula.

5. A transgenic mouse harboring a heterozygous mutation in its endogenous Alpha Hemoglobin Stabilizing Protein (AHSP) gene wherein said mouse exhibits AHSP haploinsufficiency and has an elevated reticulocyte count.

6. The transgenic mouse of claim 5, wherein said mouse is fertile and transmits said mutation to its offspring.

7. The transgenic mouse of claim 5, wherein said mutation has been introduced into an ancestor of said mouse at an embryonic stage following microinjection of embryonic stem cells into a mouse blastocyst.

8. The transgenic mouse of claim 5, wherein said mutation has been introduced into an ancestor of said mouse at an embryonic stage following co-incubation of embryonic stem cells with a fertilized egg or morula.

9. A method for screening for therapeutic agents which affect Alpha Hemoglobin Stabilizing Protein (AHSP) activity, comprising:
a) administering a test compound to the transgenic mouse of claim 1;
b) assessing said mouse for an alteration in an Alpha Hemoglobin Stabilizing Protein (AHSP) activity.

10. The method of claim 9, wherein said activity is selected from the group consisting of α-hemoglobin binding, and α-hemoglobin synthesis.

11. A method for assessing the activity of a compound useful for the treatment and/or prevention of an AHSP related disorder, comprising:
a) providing at least one Alpha Hemoglobin Stabilizing Protein (AHSP) knock-out mouse;
b) administering a test compound to said mouse; and
C) assessing said mouse for inhibition of said AHSP related disorder.

12. The method of claim 11, said method further comprising administration of said test compound to a control mouse to assess toxicity of said test compound.

13. The method of claim 11, wherein said disorder is selected from the group consisting of α-thalassemia, β-thalassemia, anemia, spongiform encephalopathy, prion disease, and Alzheimer's disease.

14. A method of diagnosing an Alpha-Hemoglobin Stabilizing Protein (AHSP) related disorder in a test subject, wherein a sample from said test subject is analyzed by a method selected from the group consisting of:
a) a method employing a specific binding member capable of binding to a AHSP nucleic acid sequence, the specific binding member comprising nucleic acid hybridizable with the AHSP sequence; and
b) a method of determining the presence, in a sample from a test subject, of a polypeptide encoded by the AHSP nucleic acid and, if present, determining the expression level; and
c) a method wherein at least one antibody domain with specificity for an epitope selected from the group consisting of a native AHSP nucleic acid sequence epitope, or a polypeptide epitope, the specific binding member being labeled so that binding of the specific binding member to its binding partner is detectable; and
d) a method of PCR amplification involving one or more primers based on AHSP gene sequence to screen for an decrease in AHSP expression in a sample from a test subject; and
e) a method of determining the presence, in a sample from a test subject, of a polypeptide encoded by the AHSP nucleic acid and, if present, determining the presence of mutations of the AHSP nucleic acid.

15. The method of claim 14, wherein said disorder is selected from the group consisting of α-thalassemia, β-thalassemia, anemia, spongiform encephalopathy, prion disease, and Alzheimer's disease.

16. The method of claim 15, wherein said disorder is bovine spongiform encephalopathy.

17. A method of screening for compounds which modulate the activity of an AHSP polypeptide, the method comprising contacting at least one test compound with the AHSP polypeptide in a reaction medium, testing the activity of the treated AHSP polypeptide and comparing that activity with the activity of native, untreated AHSP polypeptide in a comparable reaction medium.

18. A compound identified by the method of claim 11 or 17, wherein said compound is a fragment of AHSP, or a small molecule which mimics AHSP activity.

19. A method of treating, or ameliorating symptoms of an AHSP related disorder comprising over expressing an AHSP encoding nucleic acid molecule in the cells or body fluid of a patient having said disorder.

20. The method of claim 19, wherein said disorder is selected from the group consisting of α-thalassemia, β-thalassemia, anemia, spongiform encephalopathy, prion disease, and Alzheimer's disease.

21. A method of treating, or ameliorating symptoms of an AHSP related disorder comprising administering the compound of claim 18 to the cells or body fluid of a patient having said disorder.

22. The method of claim 21, wherein said disorder is selected from the group consisting of α-thalassemia, β-thalassemia, anemia, spongiform encephalopathy, prion disease, and Alzheimer's disease.

23. A method for producing alpha hemoglobin stabilizing protein (AHSP)-specific antibodies, comprising:
a) immunizing an AHSP knock out mouse with an immunogenic amount of AHSP or fragments thereof;
b) harvesting serum from said mouse; and
c) screening said serum for antibodies immunoreactive to AHSP.

24. An antibody preparation produced by the method of claim 23.

25. A method for producing alpha hemoglobin stabilizing protein (AHSP)-specific antibodies, comprising:
a) immunizing an AHSP knock out mouse with an immunogenic amount of AHSP or fragments thereof;
b) harvesting the spleen of said mouse and fusing said spleen cells with a myeloma cell line containing a mutation to facilitate isolation of fused spleen/myeloma cells;
c) culturing said fused cells in media containing a selection agent wherein fused cells grow, and non-fused cells are killed;
d) screening said media from cells surviving in the presence of said selection agent, for the presence of antibodies immunoreactive to AHSP; and
e) optionally isolating said antibody.

26. A monoclonal antibody produced by the method of claim 25.

27. A kit comprising one or more molecules for detecting AHSP expression, said molecules being optionally detectably labeled, and said molecules being selected from the group consisting of nucleic acid molecules having sequences corresponding to a portion of an AHSP nucleic acid sequence for use in amplifying a nucleic acid comprising an AHSP nucleic acid sequence, and antibodies which specifically bind to a portion of the AHSP protein.

28. A transgenic mouse characterized by overexpression of an Alpha Hemoglobin Stabilizing Protein (AHSP) gene.

29. The transgenic mouse of claim 1, said mouse also having a heterozygous null mutation in beta major and minor globulin genes on a single chromosome, said mouse exhibiting at least one characteristic selected from the group consisting of low hematocrit level, and increased red cell distribution width (RDW).

30. A method for assessing the activity of compounds useful for the treatment and/or prevention of an AHSP related disorder, comprising:
a) providing mice as claimed in claim 29;
b) administering a test compound to the mice of step (a); and
C) assessing said mice for inhibition of said AHSP related disorder.

31. The method of claim 30, said method further comprising administration of said compound to control mice to assess toxicity of said test compound.

32. A compound identified by the method of claim 30.

33. A method of treating, or ameliorating symptoms of an AHSP related disorder comprising administering the compound of claim 32 to the cells or body fluid of a patient having said disorder.

34. The transgenic mouse of claim 1, said mouse also having a homozygous null mutation in at least one alpha globulin gene, said mouse exhibiting at least one characteristic selected from the group consisting of low hematocrit level, high reticulocyte count, decreased mean corpuscular volume (MCV), and increased red cell distribution width (RDW).

35. A method for assessing the activity of compounds useful for the treatment and/or prevention of an AHSP related disorder, comprising:
a) providing mice as claimed in claim 34;
b) administering a test compound to the mice of step (a); and
C) assessing said mice for inhibition of said AHSP related disorder.

36. The method of claim 35, said method further comprising administration of said compound to control mice to assess toxicity of said test compound.

37. A compound identified by the method of claim 35.

38. A method of treating, or ameliorating symptoms of an AHSP related disorder comprising administering the compound of claim 37 to the cells or body fluid of a patient having said disorder.
